(19) United States
(12) Patent Application Publication
(10) Pub. No.: US 2011/0082424 A1
(43) Pub. Date: Apr. 7, 2011

(54) METHOD AND APPARATUS FOR BONE GRAFT INSERTION

(76) Inventors: Michael P. Barnhouse, Wilmington, NC (US); Frederic Charles Feiler, JR., Wilmington, NC (US); Walter Scott Hill, Southport, NC (US)

(21) Appl. No.: 12/572,856

(22) Filed: Oct. 2, 2009

(51) Int. Cl. A61M 5/36; A61M 5/00

(52) U.S. Cl. 604/125; 604/187; 604/506

(57) ABSTRACT

An apparatus for loading a bone graft material includes a preloading assembly, and a dispenser assembly. A method of dispensing bone graft material includes loading bone graft material into the preloader, compacting the material and transferring the compacted bone graft material into the cannula. The compacted bone graft material is vented to release unwanted or undesirable air, vapor or other gases from the bone graft material. The bone graft material is dispensed after the cannula is placed in a desired or selected position with respect to an intervertebral space.

### CROSS REFERENCE TO RELATED APPLICATIONS

The present application claims benefit and priority to U.S. Prov. Pat. Appl. No. 61/195,022 entitled BONE GRAFT INSERTION SYSTEM, filed on Oct. 2, 2008 which is hereby incorporated by reference into the present disclosure. A Patent Cooperation Treaty application assigned Ser. No. PCT/US2009/______, entitled METHOD AND APPARATUS FOR BONE GRAFT INSERTION, filed on Oct. 2, 2009, and having identical inventorship is also hereby is incorporated by reference into the present disclosure in its entirety.

### BACKGROUND

1. Field

The present disclosure relates generally to medical procedures and in particular, minimally invasive medical procedures. One application of the present teachings is in providing therapy to adjacent spinal vertebrae. More specifically, one application uses instrumentation systems that facilitate the safe and effective deployment and placement of augmentation media, such as bone growth materials or bone graft materials, via an aligned, percutaneous access and approach, to facilitate fixation, relieve lower back pain, and possibly improve disc health and prevent progression or transition of disease.

2. Background of the Related Art

Within the next decade, more than 70 million people will join the ranks of seniors and in an aging population chronic lower back pain affects both workforce productivity and health care expense. Conservative methods of treatment include bed rest, pain and muscle relaxant medication, physical therapy or steroid injection. Upon failure of conservative therapy, spinal pain has been treated by surgical interventions. Currently over 500,000 surgical procedures are performed annually in the United States in an attempt to alleviate persistant lower back pain. Generally, it is believed that 75% of cases are associated with degenerative disc disease, where the intervertebral disc of the spine suffers reduced mechanical functionality. Among surgical interventions, and based on an assessment of the patient's age, degree of disc degeneration, and prognosis, procedures that alleviate severe degenerative joint pain, or osteoarthritis, often comprise discectomy, fixation, and subsequent spinal fusion. Spinal fusion, or arthrodesis, causes the vertebrae above and below the disc to grow solidly together and form a single, solid piece of bone.

As means to enhance and facilitate osseous fusion and to repair or reconstruct vertebral bone, the introduction of various bone graft materials into the (at least partially de-nucleated) intervertebral disc space is generally an integral part of these procedures. Functions of bone graft materials include promoting osseous in-growth and bone healing, providing a structural substrate for these processes, or even serving as a vehicle for direct antibiotic delivery.

Autografts are used, but the amount of available autograft is limited in terms of locally recoverable volume, and by morbidity concerns related to autograft harvesting from a patient's other anatomical areas (e.g., iliac crest; other vascularized or non-vascularized autogenous cortical, cancellous, or corticocancellous bone; aspirated and/or enriched bone marrow stromal cells). Allograft (e.g., mineralized or de-mineralized; donor or cadaver) can “extend” autograft, but limitations include risk of disease transmission, or limitations with respect to biological properties and mechanical properties. The need for autograft substitutes has led to the development of a wide variety of synthetic substitute materials, in part based on the experiential preferences of individual surgeons. These bone graft materials vary with respect to composition, biologic properties, mechanical strength, physical properties and appearance (e.g., texture; particulate/granule size if block or segments of bone versus morcelized chips, or pieces of collagen sponge, or viscosity if introduced as a paste), and radiographic appearance (which is significant in that as post-operative radiographic studies may be performed periodically to determine new bone formation, that is, the progress of fusion, as creeping substitution allows for gradual graft resorption with osseous ingrowth and as distinguished from residual bone graft material or recurrent disease).

In the context of the present disclosure, it will be understood that as used herein the term “bone graft material” is inclusive of any suitable native or synthetic substrate or substitute/extender materials which promote or stimulate the formation of bone, e.g., osteogenic (such as autograft, aspirated or enriched bone marrow osteoprogenitor cells), osteoconductive (such as hydroxyapatite), or osteoinductive (such as demineralized bone matrix or selected bone morphogenetic proteins) materials, or any combination thereof, and/or in combination with resorbable polymers; growth factors; ceramics, or bone cements and the like.

Insertion or deployment of bone graft material into the into the intervertebral disc space is an important step in the spinal fusion procedure. However, it has also been observed that complications can occur during the insertion of bone graft materials when some bone graft insertion tools are used.

Specifically, precipitous drops in patients' blood pressure, venous emboli, including air emboli, and echogenic material flowing through heart ventricles have been clinically observed, which are believed to be attributable at least in part to a combination of factors such as the volume of bone graft material and rate at which this material is delivered, and the concomitant introduction of compressed air, (i.e., air which was retained within the bone graft material and in the bone graft insertion tool) and then conveyed (with some force) into the spine during bone graft material deployment. That is, if too great a volume of bone graft material is delivered in situ too quickly and with too much accompanying air and pressure (in part, an inherent result of bone graft material inserter design), adverse events may occur. In contrast, the device system and assemblies described in this disclosure (below) offer significant advantages in overcoming this shortcoming.

### SUMMARY OF THE INVENTION

An improved materials delivery system includes a pre-loading assembly and a dispenser or inserter assembly for introducing material along an access path to a treatment site. The inserter assembly is adapted to be introduced percutaneously through tissue to an access point on the spine in a minimally invasive, low trauma manner, to provide therapy to the spine following nucleectomy. The system and assemblies are designed to accommodate the methods of loading and deploying of a wide variety of bone graft materials substantially without the concomitant introduction of air, vapor or other gases, such as compressed air, while governing the volume and rate of said bone graft material delivery.

This summary is meant to provide an introduction to the concepts that are disclosed within the specification without being an exhaustive list of the many teachings and variations upon those teachings that are provided in the extended discussion within this disclosure. Thus, the contents of this summary should not be used to limit the scope of the claims. Other systems, methods, features and advantages of the disclosed teachings will be or will become apparent to one with skill in the art upon examination of the figures and detailed description. It is intended that all such additional systems, methods, features and advantages be included within the scope of and be protected by the claims.

The present disclosure teaches an apparatus for dispensing bone graft material. In one aspect, the apparatus can include a cannula and a dispenser rod. The cannula includes a proximal end and a distal end. The cannula also includes an inner surface defining a lumen extending between the proximal end and the distal end of the cannula. The lumen may extend substantially along a longitudinal axis of the cannula. The cannula may also include a guide extending along at least a portion of the cannula inner surface. At least one portion of the guide may include a proximal end stop and a distal end stop. The distal end stop positioned toward a distal end of the cannula from the proximal stop. The cannula may define a vent positioned between the distal end stop of the guide and the distal end of the cannula. The vent is communication with the lumen of the cannula and is adapted to release gas from the lumen. The dispenser rod defines an outside diameter that is smaller than the inside diameter of the lumen. The dispenser rod may further include a projection dimensioned to slidably engage the guide in the cannula. The dispenser rod includes a distal end and a proximal end. At least one portion of the guide may extend along at least substantially parallel to the longitudinal axis of the cannula. The guide of the cannula may extend through the inner surface of the cannula to form a slot. The projection may be slidably received in the slot. The projection may extend into the slot with the end of the projection located between the lumen and the outer surface of the cannula. The guide may be configured as a channel on the inner surface of the cannula and at least a portion of the projection may be slidably received within the channel. The guide may define at least one intermediate stop between the proximal end stop and the distal end stop. The distal end of the cannula may be beveled. The dispenser rod may include a piston secured to the distal end of the dispenser rod. The piston may be substantially cylindrically shaped and have a proximal end, a distal end, and an outside diameter that is smaller than the inside diameter of the lumen of the cannula. The lumen may define a round cross sectional shape in a transverse plane. The piston may define a dispensing vent. The distal end of the dispenser rod may be positioned within the lumen near one edge of the vent, when the projection of the dispenser rod is positioned against the proximal end stop of the guide of the cannula. The distal end of the dispenser rod may be positioned within the lumen and covering the vent, when the projection of the dispenser rod is positioned against the proximal end stop of the guide of the cannula. The volume of the lumen between the distal end of the dispenser rod when in a load position, and the distal end of the cannula may define a dispensing volume of bone graft material. The cannula may include graduation marks in the cannula that correspond to volume levels associated with the portion of the cannula between the distal end of the cannula and the distal end of the dispenser rod. The guide may include at least one intermediate stop. The guide may include a plurality of intermediate stops between the proximal end stop and the distal end stop. The intermediate stops may correlate to volumes in the lumen between the distal end of the cannula and the distal end of the dispenser rod.

The present disclosure also teaches a kit for loading a bone graft material. In one aspect, the kit may include a preloading assembly and a preloader rod. In an aspect, the preloader assembly may include a body defining a first end, a second end. The body may further define a plurality of elongated passages extending between the first end and the second end. A first end cap may be secured to a first end of the body. The first end cap may extend over at least one of the plurality of elongated passages. The first end cap can define a loading opening. The loading opening may be positionable over at least one of the plurality of openings into the elongated passages in the body and may be generally adapted to receive a bone graft material. The opening in the second end cap may allow the transfer the bone graft material into at least one of the plurality of elongated passages. A second end cap may be movably secured to a second end of the body. The second end cap can define an extraction opening. The second end cap may be movable between at least one closed position and at least one open position with respect to at least one of the plurality of elongated passages. The extraction opening in the at least one open position may be in communication with at least one of the plurality of elongated passages. In another aspect, the preloader rod is dimensioned to fit in a portion of the at least one of the plurality of elongated passages. The preloader assembly may have at least two elongated passage that define a circular transverse cross-section along at least a portion of a length of the at least two elongated passages. At least a portion of the body may be formed from a translucent material. The translucent material may extend along at least a portion of the length of at least one of the plurality of elongated passages. The translucent material adapted to transmit light between an inner surface of the body defining at least one of the elongated passages and an outer surface of the body to visualize a volume of a bone graft material positioned within the passage. The body may include one or more graduations on an outer surface of the body and extending along at least a portion of the length of at least one of the plurality of elongated passages to indicate the volume of bone graft material in the elongated passages. The preloader rod may define an outside diameter that slidably engages an inside diameter of at least one of the plurality of elongated passages. A plunger may be secured to a distal end of the preloader rod, the plunger defining an outside diameter adapted to slidably engage an inside diameter of the at least one of the plurality of elongated passages of the body. The plunger of the preloader rod further may define a plunger vent adapted to vent a gas between a first plunger end and a second plunger end. The plunger may be cylindrically shaped and defines a longitudinal axis. The plunger vent may be configured as a groove along an outer plunger surface of the plunger between the first plunger end and the second plunger end. The first end cap may define a funnel adjacent to the loading opening, the funnel adapted to guide the bone graft material into the loading opening. The first end cap may be rotatably secured to the first end of the body, the first end cap being rotatable with respect to the first end of the body such that the loading opening in the first end cap can be aligned with at least one of the plurality of elongated passages of the body. The extraction opening in the second end cap may define a cylindrical shape along at least a portion of an extraction opening length, the cylindrical shape having substantially the same inner diameter as the inner diameter of the at least one passage of the body, the extraction opening of the second end cap substantially aligning with the at least one passage of the body in the open position. The second end cap may be rotatably movable about a longitudinal axis of the body between the open position and the closed position.

The present disclosure also teaches a method for dispensing a bone graft material. In one aspect, the method may include the step of placing the bone graft material into an elongated chamber defined in a preloading assembly. The method may also include the step of compacting the material in the chamber. The method may also include the step of transferring the compacted material in the chamber to a volume in a distal end of a cannula of a dispenser apparatus. The method may also include the step of venting compressed air in the cannula from the compacted material through a vent in the cannula. The method may also include the step of positioning the distal end of the cannula to a selected position. The method may also include the step of dispensing at least a portion of the material to be dispensed by moving a dispenser rod through the cannula. In certain aspects, the method may also include the step of engaging a projection of a dispenser rod into a guide defined in the cannula to provide at least a distal end stop, and the dispensing further comprising advancing the dispenser rod distally through the lumen of the cannula until the projection contacts the distal end stop. The method may also include the step of engaging a projection of a dispenser rod into a guide defined in the cannula. The method may also include the step of providing at least an intermediate stop and a distal end stop positioned along the guide. The method may also include the step of dispensing further comprising advancing the dispenser rod distally through the lumen of the cannula until the projection contacts the intermediate stop to temporarily stop the longitudinal advancing of the dispenser rod. The method may also include the dispensing step further including rotating the dispenser rod about a longitudinal axis of the dispenser rod to disengage the projection from the intermediate stop. The method may also include the dispensing step further including advancing the dispenser rod distally through the lumen of the cannula until the projection contacts the distal end stop. The method may also include the compacting step further including using a preloader rod to compact the material to substantially remove air vapor from the material. The method may also include the materials being a slurry of bone graft material. The method may also include the step of dispensing step further including compacting the material in a chamber in the preloading assembly includes using a preloader rod to compact the material to a selected volume. The method may also include the step of compacting the material in a chamber in the preloading assembly using a preloader rod to compact the material to a selected volume, so that a selected dosage of material is associated with the chamber in the preloader. The method may also include the step of placing the material into the chamber of the preloading assembly includes aligning a funnel-shaped opening in a first end cap with the chamber of the preloader assembly. The method may also include the step of compacting the material in a chamber in the preloading assembly includeing passing a preloader rod to compact the material through the funnel shaped opening in the first end cap. The method may also include using a preloader assembly that includes a second end cap positioned at a second end of the preloader assembly in a closed position, wherein the second end cap closes the chamber in the preloading assembly. The method may also include the step of transferring the compacted material in the chamber to a volume in a distal end of a cannula of a dispenser apparatus. The transferring step including moving the second end cap to an open position with respect to the chamber in the preloader assembly. The transferring step also including moving the cannula to a transfer position with respect to the second end cap. The transferring step also including pushing the compacted material from the chamber into the cannula with the preloader rod. The transferring step also including pushing the compacted material into the cannula includes venting air from the compacted material. The method may also include the step of using an elongated passage of the preloader assembly that is an elongated cylindrical. The method may also include the step of using a second end cap with an opening that is a cylinder having substantially the same inner diameter as the inner diameter of the chamber. The method may also include using a cannula that defines an inside diameter substantially the same as the inside diameter of the opening in the second end cap.

The present disclosure teaches an apparatus for loading a bone graft material into an insertion cannula. In one aspect, the apparatus includes a preloading assembly and a dispenser rod. The preloader assembly may include a body defining a first end, a second end. The body may further define a plurality of elongated passages extending between the first end and the second end. A first end cap may be secured to a first end of the body. The first end cap may extend over at least one of the plurality of elongated passages. The first end cap can define a loading opening. The loading opening may be positionable over at least one of the plurality of openings into the elongated passages in the body and may be generally adapted to receive a bone graft material. The opening in the second end cap may allow the transfer the bone graft material into at least one of the plurality of elongated passages. A second end cap may be movably secured to a second end of the body. The second end cap can define an extraction opening. The second end cap may be movable between at least one closed position and at least one open position with respect to at least one of the plurality of elongated passages. The extraction opening in the at least one open position may be in communication with at least one of the plurality of elongated passages. In another aspect, the preloader rod is dimensioned to fit in a portion of the at least one of the plurality of elongated passages. The preloader assembly may have at least two elongated passage that define a circular transverse cross-section along at least a portion of a length of the at least two elongated passages. At least a portion of the body may be formed from a translucent material. The translucent material may extend along at least a portion of the length of at least one of the plurality of elongated passages. The translucent material adapted to transmit light between an inner surface of the body defining at least one of the elongated passages and an outer surface of the body to visualize a volume of a bone graft material positioned within the passage. The body may include one or more graduations on an outer surface of the body and extending along at least a portion of the length of at least one of the plurality of elongated passages to indicate the volume of bone graft material in the elongated passages. The preloader rod may define an outside diameter that slidably engages an inside diameter of at least one of the plurality of elongated passages. A plunger may be secured to a distal end of the preloader rod, the plunger defining an outside diameter adapted to slidably engage an inside diameter of the at least one of the plurality of elongated passages of the body. The plunger of the preloader rod further may define a plunger vent adapted to vent a gas between a first plunger end and a second plunger end. The plunger may be cylindrically shaped and defines a longitudinal axis. The plunger vent may be configured as a groove along an outer plunger surface of the plunger between the first plunger end and the second plunger end. The first end cap may define a funnel adjacent to the loading opening, the funnel adapted to guide the bone graft material into the loading opening. The first end cap may be rotatably secured to the first end of the body, the first end cap being rotatable with respect to the first end of the body such that the loading opening in the first end cap can be aligned with at least one of the plurality of elongated passages of the body. The extraction opening in the second end cap may define a cylindrical shape along at least a portion of an extraction opening length, the cylindrical shape having substantially the same inner diameter as the inner diameter of the at least one passage of the body, the extraction opening of the second end cap substantially aligning with the at least one passage of the body in the open position. The second end cap may be rotatably movable about a longitudinal axis of the body between the open position and the closed position.

### BRIEF DESCRIPTION OF THE DRAWINGS

FIG. 1 illustrates a perspective view of the apparatus for dispensing or insertion of bone graft material, according to one example.

FIG. 2 illustrates an end view of the body of the preloading assembly, according to one example.

FIG. 3 illustrates a side view of a second end cap, according to one example

FIG. 4 illustrates the dispenser rod in a load position where the projection is positioned in the proximal end stop of the guide of the cannula of the dispenser assembly, according to one example.

FIG. 4A illustrates the dispenser rod in a load position where the projection is positioned in the proximal end stop of the guide of the cannula of the dispenser assembly and the plunger covers the vent opening, according to one example.

FIG. 5 illustrates a dispenser system, according to another example.

FIG. 6A and FIG. 6B are close up cross sectional views of a portion of the dispenser rod showing the projection, according to one example.

FIG. 7 illustrates a side view of a dispenser rod having a threaded shaft, according to one example.

FIG. 8 illustrates a close-up cross sectional view of the distal end of the dispenser rod within the cannula, according to one example of the disclosure.

FIG. 9 illustrates a flow chart for a method for dispensing bone graft material, according to one example.

FIG. 10 illustrates a cross-sectional side view of the preloader rod positioned for insertion into the loading opening, according to one example.

FIG. 11 illustrates a cross-sectional side view of the preloader rod in contact with the bone graft material, according to one example.

FIG. 12 illustrates a cross-sectional side view of the preloader rod after it has been passed through the loading opening and into the opening of the body of the preloading system, according to one example.

FIG. 13 illustrates a cross sectional view of the preloader assembly having a chamber or elongated passage containing compacted bone graft material with the second end cap 400 in a closed position, according to one example.

FIG. 14 illustrates a cross-sectional side view showing the second end cap in the open position with the distal portion of the dispenser system engaged with the extraction opening, according to one example.

FIG. 15 illustrates a cross sectional side view showing the preloader rod pushing the volume of bone graft material through the second end cap and into distal portion of the dispenser system as engaged with the extraction opening, according to one example.

FIG. 16 illustrates a top view of the vent and the distal end of the dispenser rod when the dispenser rod is in the loading position, according to one example.

FIG. 17 illustrates a cross sectional side view of the distal end of the dispenser system filled with a volume of compacted bone graft material, according to one example.

FIG. 18 illustrates a cross sectional side view of the dispenser system having the distal end of the dispenser system positioned within the intervertebral cavity and having partially dispensed a portion of compacted bone graft material, according to one example.

FIG. 19 illustrates a perspective view of a cannula, according to one example.

FIG. 19A illustrates a cross sectional side view of the dispenser rod and the cannula, according to one example.

FIG. 19B illustrates a cross sectional side view of the dispenser rod and the cannula, according to another example.

All Figures are illustrated for ease of explanation of the basic teachings of the present disclosure only; the extensions of the Figures with respect to number, position, relationship and dimensions of the parts to form the examples will be explained or will be within the skill of the art after the following description has been read and understood. Further, the exact dimensions and dimensional proportions to conform to specific force, weight, strength, flow and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they will typically be utilized by a physician or other user who is treating or examining a patient with apparatus in accordance with the present disclosure.

### DETAILED DESCRIPTION

The present disclosure provides apparatus and methods for use in the introduction of bone graft materials. The figures generally illustrate examples of apparatus including aspects of the present disclosure. The particular exemplary embodiments of the apparatus and methods illustrated in the figures have been chosen for ease of explanation and understanding of various aspects of the present disclosure. These illustrated examples are not meant to limit the scope of coverage but instead to assist in understanding the context of the language used in this specification and the appended claims. Accordingly, variations of the disclosed and methods for use in the introduction of bone graft materials different from the illustrated examples may be encompassed by the appended claims.

The examples described in detail below are used to introduce or dispense bone graft material as part of spinal fusion procedure, also known as an arthrodesis. There are many steps and variations of spinal fusion. The method and apparatus described and claimed below relates to the portion of the procedure for introducing or dispensing bone graft material. Very briefly, the basic steps of a typical spinal fusion procedure include removal or denucleation of a portion of at least one intervertebral disc space, fixation of the involved vertebrae, and introducing bone graft material into the intervertebral disc space. The bone graft material may promote bone growth in the intervertebral space. The result can be that the involved vertebrae grow solidly together. One method and apparatus for introducing the bone graft material is set forth in co-pending and commonly assigned U.S. patent application Ser. No. 10/971,775 which is entitled “Method and Apparatus for Introducing Material Along an Access Path to a Treatment Site”.

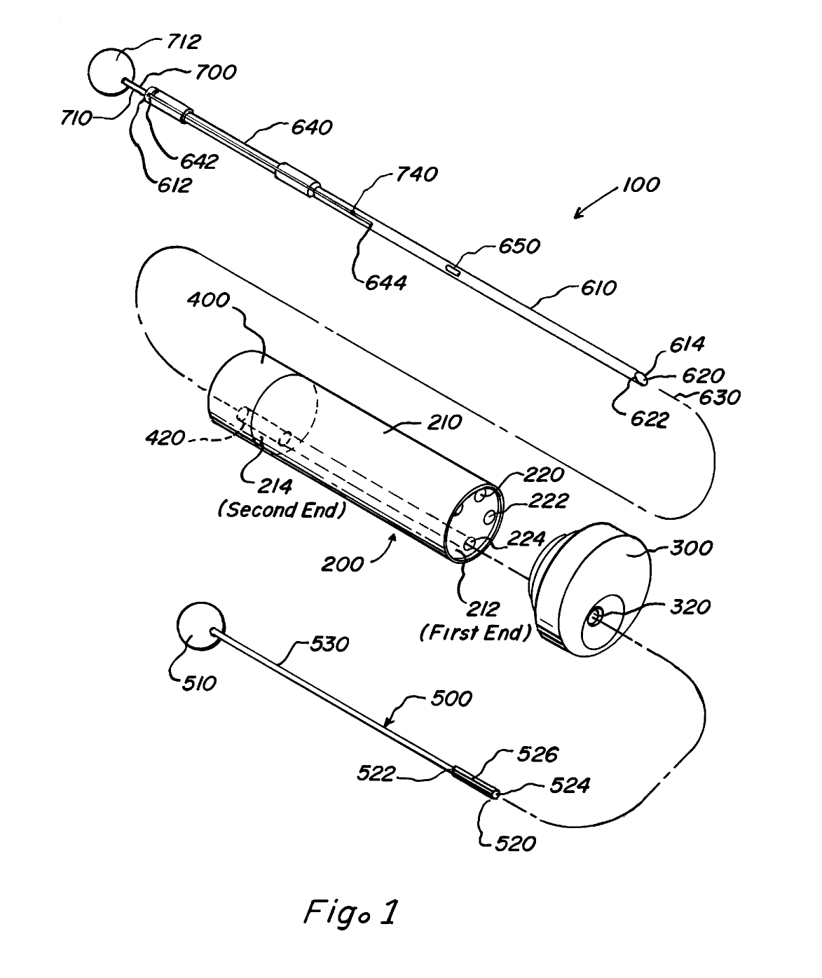

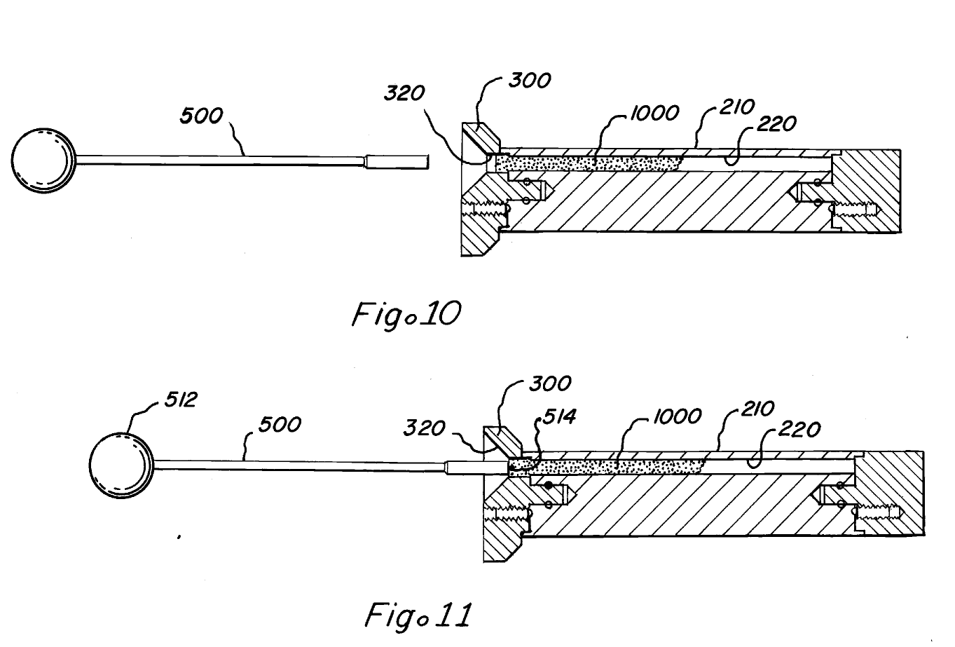

FIG. 1 is a perspective view of the apparatus for dispensing or insertion of bone graft material. Two or more of illustrated apparatus may be combined as a system or kit 100. The system 100 may include two or more of a preloader assembly 200, a preloader rod 500, and an insertion assembly 600. The insertion assembly 600 generally includes a cannula 610 and a dispenser rod 700. As a brief overview, bone graft material 1000 (shown in FIGS. 10-18) is loaded into the preloader assembly 200 and compacted with the preloader rod 500. After compacting the bone graft material, the bone graft material 1000 (shown in FIGS. 10-18) is transferred from the preloader assembly 200 to the cannula 610. The cannula 610 is then loaded with bone graft material. The cannula 610 is positioned within the patient so that the bone graft material 1000 (shown in FIGS. 10-18) can be dispensed into the intervertebral disc space. The preloader assembly 200 includes a body 210, a first end cap 300 and a second end cap 400. The various parts of the system or kit 100 will now be further detailed. The method will be detailed after the detailed description of the apparatus.

### Preloader Assembly

The preloader assembly 200 is generally configured to receive a bone graft material into an elongated preloader assembly passage, permit the compacting of the bone graft material and to transfer the bone graft material 1000 (shown in FIGS. 10-18) to the cannula 610. The preloader assembly 200 may include a body 210, a first end cap and a second end cap. The body 210 may be generally elongated and will typically define a first end 212 and a second end 214. As illustrated for exemplary purposes, the body 210 is generally configured as a cylinder. The body 210 may define one or more elongated preloader assembly passages 220, 222, 224 extending between the first end 212 and the second end 214. As shown, the body 210 of the preloader assembly 200 has three elongated passages having a substantially circular transverse cross-section along at least a portion a length of the elongated passages for exemplary purposes. The elongated preloader assembly passages 220, 222, 224 may be arranged parallel to one another and may also be parallel to the longitudinal axis of the body 210. The longitudinal axis of the body 210 may extend between the first end and the second end of the body 210. In one aspect, the elongated preloader assembly passages 220, 222, 224 may be formed in an array about the longitudinal axis of the body 210. It should be noted that there can be more or fewer passages, such as elongated passages 220, 222, 224 extending through the body 210. Most embodiments include at least two passages. There could also be an embodiment that includes only one elongated passage. It should be noted that the elongated passages 220, 222, 224 do not necessarily have to have a circular transverse cross section. In certain aspects, the elongated preloader assembly passages 220, 222, 224 could be formed in various alternative shapes as a transverse cross section, such as, for example, oval, oblong, square, triangular or the otherwise shaped as will be recognized by those skilled in the art upon review of the present disclosure. A portion or all of the body 210 may be formed from a translucent material. The translucent material may extend along at least a portion of the length of at least one of the plurality of elongated preloader assembly passages 220, 222, 224 and can be adapted to transmit light between an inner surface of the body 210 defining at least one of the elongated preloader assembly passages 220, 222, 224 and an outer surface 216 of the body 210. In certain aspects, body graduations may be provided on the side of the body 210 to permit a user to assess the volume of material being compacted in or already contained in the preloader assembly 200.

The body 210 is typically sized from between about 100 mm and about 200 mm in length, and often about 150 mm, with a body outer diameter from between about 25 mm and about 50 mm in diameter, and often about 32 mm. In general, the length of the elongated preloader assembly passages 220, 222, 224, in the body 210 is from between about 80 mm and about 160 mm in length, and often about 120 mm. The diameter of the loading chambers or elongated passages 220, 222, 224 is from between about 4 mm and about 10 mm in diameter, and often about 6 mm.

The first end cap 300 is typically secured to a first end 212 of the body 210. In certain aspects, the first end cap 300 may be removably and/or rotatably secured to the first end 212. At least a portion of the first end cap 300 may be secured over at least one of the plurality of elongated passages 220, 222, 224. The first end cap 300 may define a loading opening 320. The loading opening 320 is generally configured to permit the passage of bone graft material. The loading opening 320 is typically positionable over at least one of the plurality of openings 220, 222, 224 and may be adapted to receive a bone graft material and to transfer the bone graft material 1000 (shown in FIGS. 10-18) into at least one of the plurality of elongated passages 220, 222, 224. As generally illustrated in FIG. 1, the first end cap 300 defines loading opening 320 extending between a first end and a second end of the first end cap 300. The first end cap 300 further defines a funnel-shaped portion 322 of the loading opening 320 on a first end of the first end cap 300. The funnel shaped portion 322 being adapted to facilitate loading of the bone graft material 1000 (shown in FIGS. 10-18) into opening 320. The first end cap 300 is configured to align the loading opening 320 with at least one of the elongated passages 220, 222, 224 when the first end cap 300 is secured to the first end 212 of the body 210. The funnel 322 is adapted to guide the bone graft material into the loading opening 320. The diameter of the loading opening 320 at the surface contacting the first end 212 of the body 210 may be substantially the same as the cross sectional diameter of one or more of the elongated passages 220, 222, 224. As generally illustrated for exemplary purposes, the first end cap 300 is rotatably secured to the first end 212 of the body 210 and rotates about a longitudinal axis of the body 212 such that the loading opening 320 in the first end cap 300 can be aligned with at least one of the plurality of elongated passages 220, 222, 224 that are arranged in an array about the longitudinal axis of the body 210.

The second end cap 400 is typically movably secured to a second end 214 of the body 210. In certain aspects, the second end cap 400 may rotatable about a longitudinal axis of the body 210. The second end cap 400 can define at least one extraction opening 420. The second end cap 400 is typically configured to position the extraction opening 420 over at least one of the plurality of elongated passages 220, 222, 224 to permit the passage of a bone graft material 1000 (shown in FIGS. 10-18) from the plurality of elongated passages 220, 222, 224 through the extraction opening 420. Typically, the second end cap 400 is movable between at least one closed position and at least one open position with respect to at least one of the elongated passages 220, 222, 224. In the closed position, the second end cap 400 is adapted to permit the packing of a bone graft material 1000 (shown in FIGS. 10-18) against the portion of the second end cap 400 that closes off the elongated passage 22, 222, 224 being filled. In the open position, the extraction opening 420 is typically juxtaposed with a second end of at least one of the plurality of elongated passages 220, 222, 224. In certain aspects, the open position of the extraction opening 420 may align the longitudinal axis of the extraction opening 420 and the opening at the second end of one of the plurality of elongated passages 220, 222, 224. The second end cap 400 may have a substantially flat surface that interfaces with the second end 214 of the body. In the closed position, a portion of the substantially flat surface closes the end of the openings 220, 222, 224. In one aspect, the second end cap 400, may include a single closed position, covers all of the elongated passages 220, 222, 224. The second end cap 400 may be rotatably movable about a longitudinal axis of the body 210 to position the extraction opening 420 over the openings of the elongated passages 220, 222, 224 at the second end of the body in the open position and to position the extraction opening 420 to either side of every opening of the elongated passages 220, 222, 224 in the closed position.

The extraction opening 420 in the second end cap 400 may have a cross sectional shape that corresponds in one or both of size and shape to the cross sectional shape of the elongated passages 220, 222, 224. In certain aspects, the extraction opening 420 can define a cylindrical shape along at least a portion of the length of the extraction opening 420. In one aspect, the cylindrical shape of the extraction opening 420 may have substantially the same inner diameter as the inner diameter of the at least one elongated passage 220, 222, 224 at the surface of the second end cap 400 secured to the second end 214 of body 210. In another aspect, the extraction opening 420 may have a slightly larger inner diameter when compared to the inner diameter of the at least one passage 220, 222, 224 at the surface of the second end cap 400 secured to the second end 214 of body 210.

The extraction opening 420 of the second end cap 400 may be positioned off center with respect to the longitudinal axis of the second end cap 400 so that it may be rotatably aligned with the various elongated passage 220, 222, 224. In one aspect, the second end cap 400 may be secured in an open position or a closed position. In one particular aspect, the second end cap 400 may be adapted to integrally engage with the second end 214 of the body 210 when the extraction opening 420 is in either a defined open or a defined closed position. In this aspect, the second end cap 400 may be configured with a detent mechanism. The detent mechanism may increase resistance to further rotation of the second end cap 400 when the extraction opening 420 of is positioned at least one of an open position or a closed position.

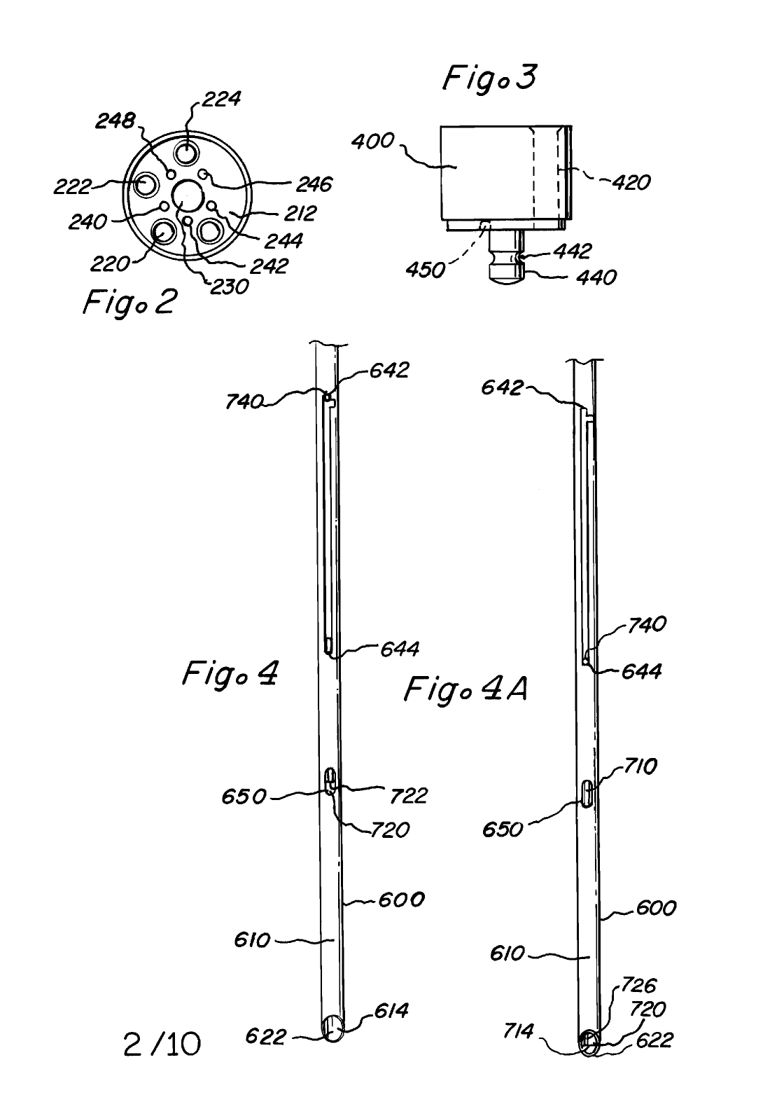

FIG. 2 shows an end view of the body 210 integrating various features of an exemplary embodiment of a detent mechanism. The second end 214 (see FIG. 1) of body 210 is shown in FIG. 2. The second end 214 includes an opening 230 which receives a connecter 440 (see FIG. 3) from the portion of the second end cap 400 adapted to engage the second end 214 of the body 210. As illustrated, the opening may be coaxial with the longitudinal axis of the body 210. The connector 440 may form the axis about which the second end cap 400 may rotate. The exemplary detent mechanism includes a set of detents 240, 242, 244, 246, 248 extending from the second surface 214 of the body 210 and a detent recess 450 shown in phantom on the surface of the second end cap 400. The detents 240, 242, 244, 246, 248 are individually positioned within the detent receiver as the second end cap 400 is rotated about the connector 440 to hold the second end cap 400 in place and yet allow the second end cap 400 to be rotated to a number of open and closed positions with respect to the chambers or elongated passages 220, 222, 224 in the body 210 of the preloader assembly 200. In certain aspects, the detents 240, 242, 244, 246, 248 may have a spherical shape and the detent recess 450 may have a concave inner surface configured to permit the detent to securely nest within the recess.

FIG. 3 shows a side view of a second end cap 400, according to an exemplary embodiment. The second end cap 400 includes an opening 420 which is a straight side walled opening for transferring bone graft material 1000 (shown in FIGS. 10-18) into the opening 420 from one of the aligned elongated passages 220, 222, 224 of the body 210 (see FIG. 1). FIG. 3 also details the connector 440. The connector 440 includes an annular notch 442 dimensioned to receive a retaining projection, not shown, extending into opening 230 of body 210. The retaining projection engages the annular notch 442 and allows for rotational movement so that the connector 440 of the second end cap is retained in opening 230. Thus, the detents 240, 242, 244, 246, 248 may be retained in the detent recess 450 to align the opening 420 with any of the elongated passages or chambers 220, 222, 224 in the body 210.

Referring to FIG. 1, the kit or system 100 may also include the preloader rod 500. The preloader rod 500 is adapted to pass through the loading opening 320 and into one of the elongated passages 220, 222, 224 and used to plunge or tamp or otherwise compact bone graft material 1000 (shown in FIGS. 10-18) in the aligned chamber or elongated passage 220, 222, 224. The preloader rod 500 is dimensioned to fit in a portion of the at least one of the plurality of elongated passages 220, 222, 224. In one embodiment, the preloader rod 500 defines an outside diameter that fits within the inside diameter of the elongated passages 220, 222, 224 as well as the cylindrical loading opening 320. As shown in FIG. 1, the preloader rod 500 may include a plunger 520 secured on an end of the preloader rod 500. The plunger 520 having outside diameter adapted to slidably engage the inside diameter of the at least one of the plurality of elongated passages 220, 222, 224 of the body 210. The preloader rod 500 may also include a preloader handle 510. The handle 310 is typically secured to an end of the preloader rod 500 opposite the end to which the plunger 520 is secured. The handle is typically configured to permit a user to manipulate it by hand and, as particularly illustrated, may be spherical in shape. The remaining portion of the preloader rod 500 or intermediate body 530 may have a smaller diameter than the plunger 520.

The plunger 520 has a proximal end 522 and a distal end 524. In certain aspects, the plunger 520 may be generally cylindrical or otherwise shaped to generally conform to the inner wall defining elongated passages 220, 222, 224. The plunger 520 of the preloader rod 500, as shown in FIG. 1, may have a plunger vent 526. The plunger vent 522 is generally adapted to allow the venting of gases or vapors between a first or proximal plunger end 522 and a second or distal plunger end 524. In one aspect, the plunger 520 may be cylindrically shaped and define a longitudinal axis. In such an embodiment, the plunger vent 526 may be configured as a groove along an outer plunger surface of the plunger 520 between the proximal plunger end 522 and the distal plunger end 524. In other embodiments, the plunger 520 can be devoid of a plunger vent 526, or can include a plunger vent 526 through the body of the plunger 520 rather than on its surface. In this aspect, the plunger vent 526 may extend as a passage through the plunger 520. In still other embodiments, the plunger vent 526 may not a linear groove or path.

In the exemplary embodiment shown in FIG. 1, the preloader handle 510 is configured as a spherical knob with a radius of curvature that matches that of the concavity in the top of the loader funnel 322 in the first end cap 300. The plunger 520 of the preloader rod 500 may be inserted through the concaved or funnel shaped top 322 of the load opening 320, through the load opening 320 and into a chamber or elongated passage, 220, 222, 224, to compresses the bone graft material 1000 (shown in FIGS. 10-18) toward the second end 214 of the passage 220, 222, 224, while expelling air and other vapors or gases. In this aspect, the plunger vent 526 in the plunger 520 facilitates air or other fluid venting.

The body 210 of the preloading assembly 200 may be fabricated from a semi-transparent polymer, such as a polysulfone, such as Udel® (Solvay Advanced Polymers), which enables visualization of the bone graft materials loading, compression, and dispensing processes using the pre-loading device assembly. The first end cap 300 and its associated loading funnel 322, and the preloader handle 510 may be fabricated (e.g., molded or machined) from any suitable (i.e., biocompatible material with adequate mechanical properties), sterilizable medical grade polymer, for example an acetyl copolymer, such as Delrin® (E. I. du Pont de Nemours and Company). The preloader rod 500 shaft and plunger 520 may be fabricated (e.g., machined, from the distal end tip and stepped down to the intermediate shaft body, i.e., said shaft is), for example, from 300 series medical grade stainless steel. The components (individually or collectively) of the pre-loading assembly 200 and the preloader rod 500 may be either disposable, or reusable.

The length and diameter of each elongated passage 220, 222, 224 collectively determine the amount of bone graft insertion material contained therein and the amount subsequently transferred to the bone graft dispenser 600 for deployment. In general, the amount of bone graft material 1000 (shown in FIGS. 10-18) dispensed from each loading chamber or elongated passage 220, 222, 224 is from between about 2 cc and about 7 cc, and often about 3 cc.

### Dispenser Assembly

Still looking at FIG. 1, the kit or system 100 for dispensing bone graft material 1000 (shown in FIGS. 10-18) may include the dispenser assembly 600. The dispenser assembly 600 includes a cannula 610 and a dispenser rod 700. The cannula 610 has a proximal end 612 and a distal end 614. The cannula 610 also includes a cannula inner surface 620 that defines a lumen 622 between the proximal end 612 and the distal end 614. The lumen 622 extends substantially along a longitudinal axis 630 of the cannula 610. A guide 640 is defined by the cannula 610 to receive a projection 740 of the dispenser rod 700. The guide 640 extends along at least a portion of the length of the lumen 622 of the cannula 610. The guide 640 may extend at least into the cannula inner surface 620 and can extend along the cannula inner surface 620 substantially parallel to the longitudinal axis 630 of the cannula 610. The guide 640 includes a proximal end stop 642, and a distal end stop 644. The proximal end stop 642 is positioned proximal to a distal end 614 of the cannula 610. The cannula 610 also defines a vent 650 extending from an outer surface of the cannula 610 and into the lumen 622. The vent 650 is typically positioned between the distal end stop 642 of the guide 640 and the distal end 614 of the cannula 610. The vent 650 is adapted to vent to release pressurized air, vapors, or other gases, from the lumen 622. The pressurized air, vapors or other gases may be introduced into the bone graft material 1000 (shown in FIGS. 10-18) during the loading process. The vent 650 shown in FIG. 1 may be a single opening that extends from the lumen 622 to the outer surface of the cannula 610. In some other embodiments, the vent 650 could include a plurality of openings. The vent 650 is typically configured to maintain the structural integrity of the cannula 610. As shown in FIG. 1, the guide 640 of the cannula 610 may extend through the outer surface of the cannula to form a slot. In FIG. 1, most of the slot is substantially straight and parallel to the axis 630 of the cannula 610. In other embodiments, the portion of the guide 640 may include one or more intermediate stop between the proximal end stop 642 and the distal end stop 644. The intermediate stops may be configured to encumber the advancing of the dispenser rod 700 along the length of the lumen 622 of the cannula 610. In certain aspects, the intermediate stops may require that the dispenser rod 700 be twisted to disengage the projection 740 from abutted intermediate stop before the dispenser rod 700 may be further advanced.

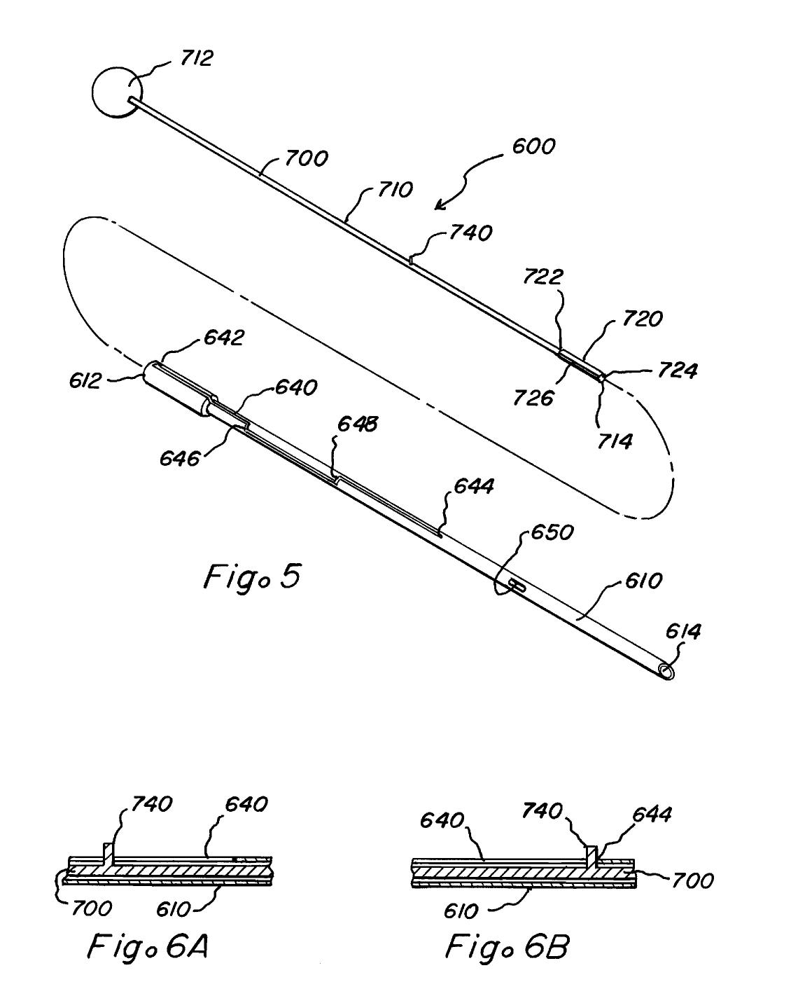

FIG. 1 also includes a dispenser rod 700. The dispenser rod includes a shaft 710 and a handle 720. The dispenser rod 710 includes a proximal end 712 and a distal end 714 (shown in FIG. 5). The dispenser rod 700 has an outside diameter that is smaller than the inside diameter of the lumen 622 of the cannula 610. The dispenser rod 700 further includes a projection 740 dimensioned to slidably engage the guide 640 in the cannula 610. The distal end 714 of the dispenser rod 700 is typically in fluid communication with the vent 650 in the cannula 610 when projection 740 is positioned against the proximal end stop 642.

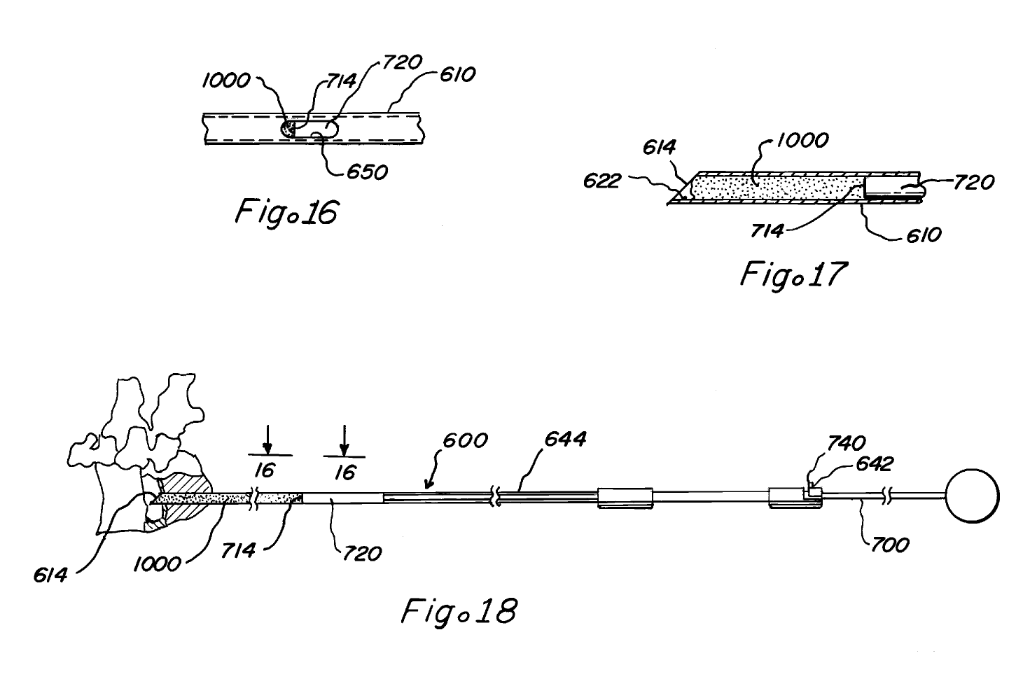

FIG. 4 shows the dispenser rod 700 in an exemplary load position where the projection 740 is positioned in the proximal end stop 642 of the guide 640 of the cannula 610. As illustrated, the tip 720 of the dispenser rod 700 may loosely engage the inner surface 620 of the cannula 610 of the lumen 622 to permit the venting of gas around the tip 720 and through the vent 650. The distal end 714, shown in FIG. 5, of the tip 720 of the dispenser rod 700 extends just distal to a distal portion of the vent 650 to allow air, vapor or other gases, to be expelled during compaction and insertion of the bone graft material 1000 (shown in FIGS. 10-18) into the distal end of the lumen 622 of the cannula 610 while not allowing significant amounts of bone graft material 1000 (shown in FIGS. 10-18) to exit the vent 650. In similar embodiments as illustrated in FIG. 16, the distal end 714 of the tip 720 of the dispenser rod 700 may be positioned just proximal to a distal portion of the vent 650 to allow venting of air, vapor or other gases. In certain aspects when the distal end 714 of the tip 720 of the dispenser rod 700 extends partially into or just distal to the vent 650, the distal end 714 substantially prevents most bone graft materials from flowing past the distal end 714 while allowing for venting of air and other gases and vapors around the tip 720 and through the vent 650 during loading.

FIG. 4A shows the dispenser rod 700 in a dispensed position where the projection 740 is positioned in the distal end stop 644 of the guide 640 of the cannula 610. In this position, the distal end 714 of the tip 720 at the end of the dispenser rod 700 extends from the distal opening of the lumen 622. In various aspects, the distal end 714 of the tip 720 may be configured to extend to various locations along, before or beyond the distal end of the cannula 610 when in the dispensed position.

FIG. 5 shows another example of a dispenser system 600. The dispenser system 600 includes a cannula 610 and a dispenser rod 700. The dispenser rod 700 includes a shaft 710 and a tip 720. The shaft 710 includes a projection 740. The projection 740 may be formed from a pin secured to and extending from the shaft 710 that may be secured within the transverse plane. The tip 720 has an external shape that substantially corresponds to internal shape of the lumen 622. As illustrated, the tip 720 is generally cylindrically shaped and defines a proximal end 722, and a distal end 724. Particularly when the shape of the tip 720 and internal shape of the lumen 622 do not permit the rotation of the tip 720 in the lumen 622, the tip may be rotatably secured to the shaft 710 to permit rotation around the longitudinal axis. The tip 720 has an outside diameter that is smaller than the inside diameter of the lumen 622. The tip 720 is attached to the distal end 714 of the dispenser rod 700. The dispenser rod 700 has a shaft 710 with an outside diameter which is smaller than the outside diameter of the tip 720. The tip 720 includes a dispenser vent 726. The dispenser vent 725 may be configured to vent air, vapors or other gases during either or both of the loading and dispensing of bone graft material 1000 (shown in FIGS. 10-18) from the cannula 610. The dispenser vent 726 may be configured as a groove along an outer tip surface of the tip 720 between the proximal end 722 and the distal end 714. In other embodiments, the tip 720 can be devoid of a dispenser vent 726, or can include a dispenser vent 726 through the body of the tip 720 rather than on its surface. In this aspect, the dispenser vent 726 may extend as a passage through the tip 720. In still other embodiments, the dispenser vent 726 may not a linear groove or path. The distal end 714 of the dispenser rod 700 is positioned within the lumen 622 near one edge of the vent, when the dispenser rod is in the proximal end stop position (the load position shown in FIG. 4). The volume of the lumen 622 between the distal end of the dispenser rod 714 when in the proximal end stop position 642, and the distal end 614 of the cannula 610 defines a dispensing volume of bone graft material. In certain aspects, the guide 640 includes at least one intermediate stop 646, 648. The intermediate stops 646, 648 may be configured as a turn, narrowing, bend, high friction material or other feature of the guide 640 or in the guide 640 that inhibits the distal movement of the dispenser rod 700 through the lumen 622 of the cannula 610 as will be recognized by those skilled in the art upon review of the present disclosure. As generally illustrated in FIG. 5, the intermediate stops 646, 648 may be configured as turns in the guide 640. The illustrated guide extends parallel to a longitudinal axis of the cannula 610 for a majority of the length and turns at a 90 degree angle to extend in the transverse plane around the cannula 610 at the intermediate stops 646, 648. This configuration of intermediate stops 646, 646 requires a user to rotate the dispenser rod 700′ around its longitudinal axis when the pin 740 contacts each intermediate stop 646, 648. Thus, among other things, it may slow the act of dispensing bone graft material 1000 (shown in FIGS. 10-18) relative to dispenser systems without such intermediate stops 646, 648. In some aspect, such as the one shown in FIG. 5, the guide 640 includes a plurality of intermediate stops 646, 648 between the end stop point 642 near the proximal end of the cannula, and the end of the guide 640 near the distal end of the cannula 614. The intermediate stop 646, 648 can correlate to volumes associated with the portion of the cannula 610 between the distal end 614 of the cannula 610 and the distal end 714 of the dispenser rod 700. The intermediate stops 646, 648 also slow down the rate that the dispenser rod 700 can be advanced since each stop point requires a directional change of the pin 740 before it can be further advanced in the guide 640.

In another aspect, the cannula 610 may includes graduation marks that correspond to volume levels associated with the portion of the cannula (volume) between the distal end 614 of the cannula 610 and the distal end 714 of the dispenser rod 700. The guide 640 may also include a tortuous path for slidably engaging the projection 740 of the dispenser rod 700. In some exemplary configurations, the guide 640 is a slot and the projection 740 is configured as a pin which extends beyond the outer diameter of the cannula 610. The pin slidably engages with the slot while the dispenser rod 700 is slidably engaged with the lumen 622. The length of the guide 640 in the cannula 610 is from between about 60 mm and about 160 mm, and often about 100 mm. The guide length and configuration serve to help gate or control the rate of insertion of the bone graft material 1000 (shown in FIGS. 10-18) by the dispenser rod 700.

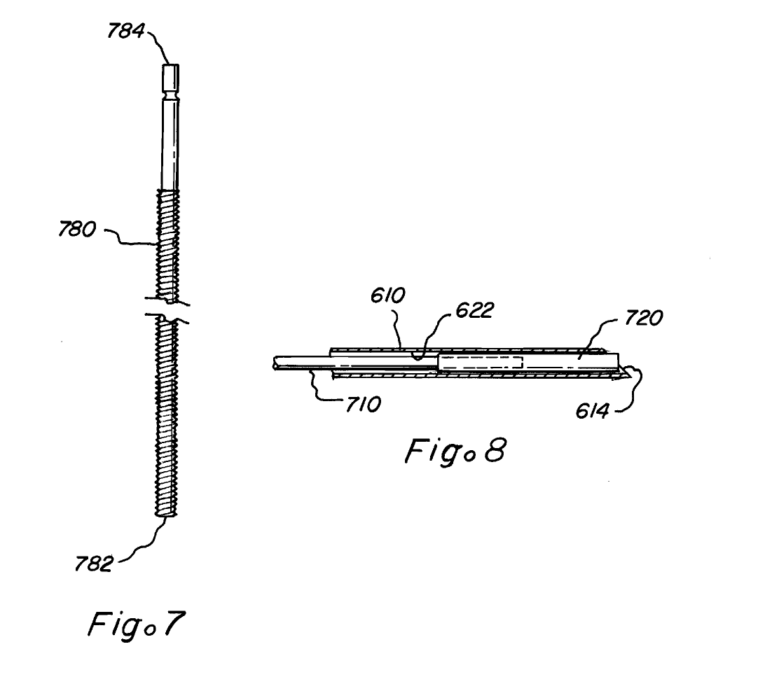

FIG. 6A and FIG. 6B are close up cross sectional views of a portion of the dispenser rod 700 showing the projection 740, according to an exemplary embodiment. The dispenser rod 700 is placed within the cannula 610. More specifically, the projection 740 is placed within the guide 640. The projection 740 extends beyond the outside surface of the cannula 610. The projection 740 is slidably engaged with the guide 640. In FIG. 6A, the projection 740 is in a position within the slot. In FIG. 6B, the projection is positioned against a stop, such as proximal end stop 642, distal end stop 644, or one of the intermediate stops 644, 646. In FIG. 6B, the projection is stopped at distal end stop 644.

FIG. 19 is a perspective view showing two alternate examples of the dispenser apparatus. FIG. 19A shows a cross sectional view of the dispenser rod and the cannula, according to one example, and FIG. 19B shows a cross sectional view of the dispenser rod and the cannula, according to another example. In both examples, the projection does not extend to the outer surface of the cannula.

As shown in FIG. 19B, a dispenser rod 1700 includes a projection 1740, having an end face. The end face can be beveled, rounded, flat or of any desired shape. The dispenser rod 1700 is placed within the cannula 1610. More specifically, the projection 1740 is placed within a guide 1640 in the cannula 1610. The projection 1740, and more specifically the end face of the projection 1740, does not extend beyond the outside surface of the cannula 1610. Rather the end face extends to a position between the outside surface of the cannula 1610 and the inner surface of the cannula 1620. The projection 1740 is slidably engaged with the guide 1640. The shape of the end face of the projection is shaped to slide smoothly and without binding in the guide 1640. The guide 1640, in the embodiment shown in FIG. 19B passes through the wall of the cannula 1610.

A further example is shown in FIG. 19A. FIG. 19A has all the same components as the example shown in FIG. 19B. The difference is that an outer sleeve 1612 is placed over the cannula 1610 outer surface. The outer sleeve 1612 and the guide 1640 make a channel or keyway. The guide 1640 or a portion of the guide 1640 is covered with the outer sleeve such that the projection 1740 and the guide 1640 will not pinch a surface or a member. The outer sleeve 1612 can be made of an opaque material which results in a blind keyway or channel. The outer sleeve can also be made of a translucent material so the position of the projection 1740 relative to the guide 1640 can be determined via visual inspection.

Returning back to FIGS. 1, 4 and 5 the distal end 614 of the cannula 610 is shown as beveled. The distal end 614 of the cannula 610 is configured to be beveled at an angle from between about 25 degrees and about 75 degrees relative to the longitudinal axis 630 of the cannula 610. Often the beveled angle is about 45 degrees. The beveling results in the distance between the proximal 612 and distal 614 ends of the cannula 610 being longer along a bottom surface of the cannula 610. The cannula 610 may be rotated to direct the beveled distal end 614 and hence the direction of deployment of the bone graft material 1000 (shown in FIGS. 10-18) during its delivery, in situ.

It is additionally noted that the presence of the guide 640 and one or more of the stops 642, 644, and intermediate stops 646, 648 must result in a cannula 610 that has sufficient wall thickness for structural strength. In some embodiments, the cannula 610 is fabricated from 300 series medical grade stainless steel. The wall thickness of the cannula 610 is from between about 0.2 mm and about 1.0 mm, and often about 0.4 mm. The length of the cannula 610 is from between about 250 mm and about 450 mm, and often about 330 mm. The inner diameter of the cannula 610 is from between about 4 mm and about 8 mm, and often about 5.6 mm. In another example, the inner diameter of the cannula is between 4 mm and 11 mm and often about 7 mm. The outer diameter of the cannula 610 is from between about 5 mm and about 11 mm, and often about 7 mm, and is determined relative to the inner diameter of a working cannula (not shown) used to provide a trans-sacral access tract in a spinal fusion procedure. The working cannula (not shown) generally is within a range of about a 9 mm inner diameter to 11 mm inner diameter and the bone graft inserter assembly 600 (see FIG. 1) is deployed through the working cannula (not shown) in order to deliver bone graft materials.

FIG. 7 shows another exemplary embodiment of a dispenser rod 780. The dispenser rod 780 includes a proximal end 782 and a distal end 784. The dispenser rod 780 also includes a threaded shaft 786. The rate of delivery of bone graft material 1000 (shown in FIGS. 10-18) is gated to an even slower rate when the dispenser rod 780 is used in a cannula 610 since the bone graft material is advanced by rotating a threaded, rotational handle (not shown).

The dispenser rod 700, in one embodiment, is fabricated from 300 series medical grade stainless steel. The handle at the proximal end 712 of the dispenser rod 700 is fabricated from a suitable, sterilizable polymer, such as an acetyl copolymer. The distal end 714 of the dispenser rod 700 includes the tip 720 at the end of the shaft 710 (see FIG. 5). The shaft 710 and tip 720, in one embodiment, is configured as a sub-assembly with an over-molded (injection molded) polymeric tip 720 on the rod shaft 710.

The polymeric tip 720 may also be press-fit onto the rod shaft 710. The tip may be fabricated from a polyvinylidene fluoride (PVDF), such as Kynar® (Elf Atochem North America, Inc) since the PVDF inserter tip reduces friction as the dispenser rod 700 is distally advanced through the cannula 610. In addition, PVDF is dimensionally stable and hydrophobic. Thus, if the device is steam sterilized, there will be no dimensional shift resulting from moisture gain, which would compromise the press-fit, mentioned above.

In general the dispenser rod 700 is from between about 275 mm and about 475 mm, and often about 355 mm in overall length, and is from between about 2 mm and about 10 mm, and often about 4 mm in diameter. The over-molded or press fit tip 720 is from between about 25 mm and about 50 mm, and often about 38 mm in length, and is from between about 3.8 mm and about 10.5 mm, and often about 5.3 mm in diameter. This diameter is dependent upon the inner diameter of the cannula 610 into which it is inserted.

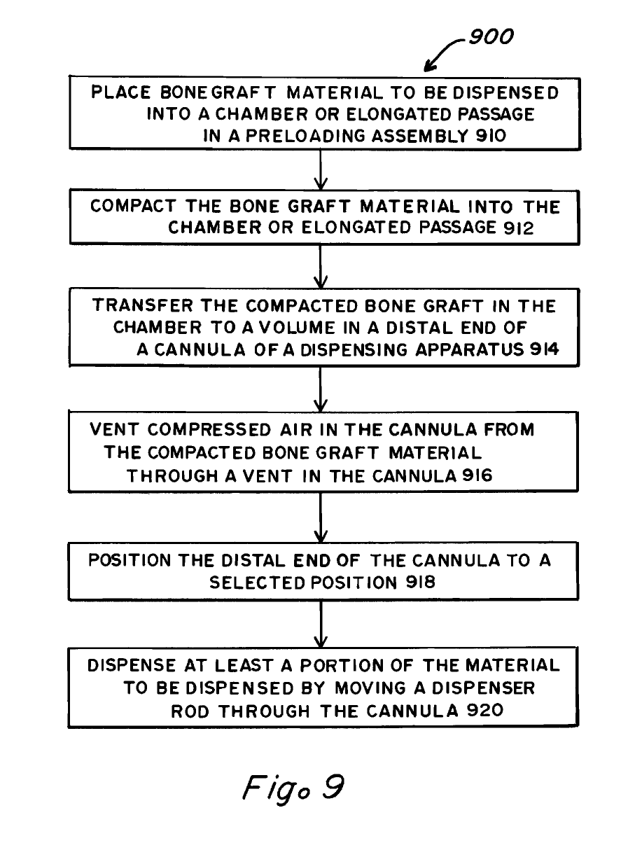

FIG. 8 is a close-up cross sectional view of the distal end 714 of the dispenser rod 700 within the cannula 610, according to an exemplary embodiment of the disclosure. The distal end 714 includes tip 720. The tip outer diameter 720 is dimensioned so that it is able to slidably engage the inner diameter of the cannula 610. Put another way, the tip 720 outer diameter is dimensioned to slide within the lumen 622 of the cannula 610.

The advantages of the instrumentation systems configurations of the present disclosure in enabling safer and effectively controlled materials delivery will become more apparent from the discussion regarding the use of the preloader assembly, preloader rod, and the dispenser assembly 600, which is detailed in the following paragraphs.

### Method of Use of Exemplary Bone Graft Material Loading & Inserter System

In one aspect of the present disclosure, there is disclosed a method of use of a bone graft materials deployment system 100 comprises a preoader assembly 200, and a dispenser assembly 600, which is introduced to the surgical site in a minimally invasive, percutaneous presacral approach and through a trans-sacral access tract.

FIG. 9 is a flow chart for a method 900 for dispensing bone graft material, according to an exemplary embodiment. The method 900 is an overview and will be further detailed in the following paragraphs. The method 900 for dispensing bone graft material includes placing bone graft material to be dispensed into a chamber or elongated passage 910 in a preloading assembly, compacting the bone graft material in the chamber or elongated passage 912, transferring the compacted bone graft material 914 in the chamber to a volume in a distal end of a cannula of a dispenser apparatus. The method 900 also includes venting compressed air in the cannula from the compacted bone graft material through a vent in the cannula 916, positioning the distal end of the cannula to a selected position 918, and dispensing at least a portion of the material to be dispensed by moving a dispenser rod through the cannula 920. The method will now be further detailed with reference to accompanying figures.

Placing the material into the chamber of the preloading assembly 910 includes aligning the opening 320 with the funnel-shaped portion 322 in a first end cap 300 with at least one of the elongated passages 220, 222, 224 or chambers of the preloader assembly 200 (see FIG. 1). Initially, the surface of the first end cap 300 opposite the funnel portion 322 of the opening 320 is placed onto the first end 212 of the body 212, and rotated to align the loading opening 320 with at least one of the elongated passages 220, 222, 224 or chambers of the body. The second end cap 400, which is also referred to as the channel selector cap, is attached to the second end 214 of the body 210 and rotated to the closed position. The second end cap 400 then closes off the ends of the elongated passages 220, 222, 224. The selected chamber or elongated passage 220, 222, 224 is loaded with bone graft material through the funnel and loading opening 320. The selected elongated passage or chamber is filled by introducing the bone graft materials selected by the surgeon into the concavity associated with the funnel portion 322 of the loading opening 320.

The result is seen in FIG. 10 with the selected elongated passage 220 and the loading opening 320 partially filled with bone graft material 1000. As shown in FIG. 10, the preloader rod 500 is then positioned for insertion into the loading opening 320

FIG. 11 shows the preloader rod 500 in contact with the bone graft material 1000. Specifically, the distal end 714 of the preloader rod 500 is placed into contact with the bone graft material 1000.

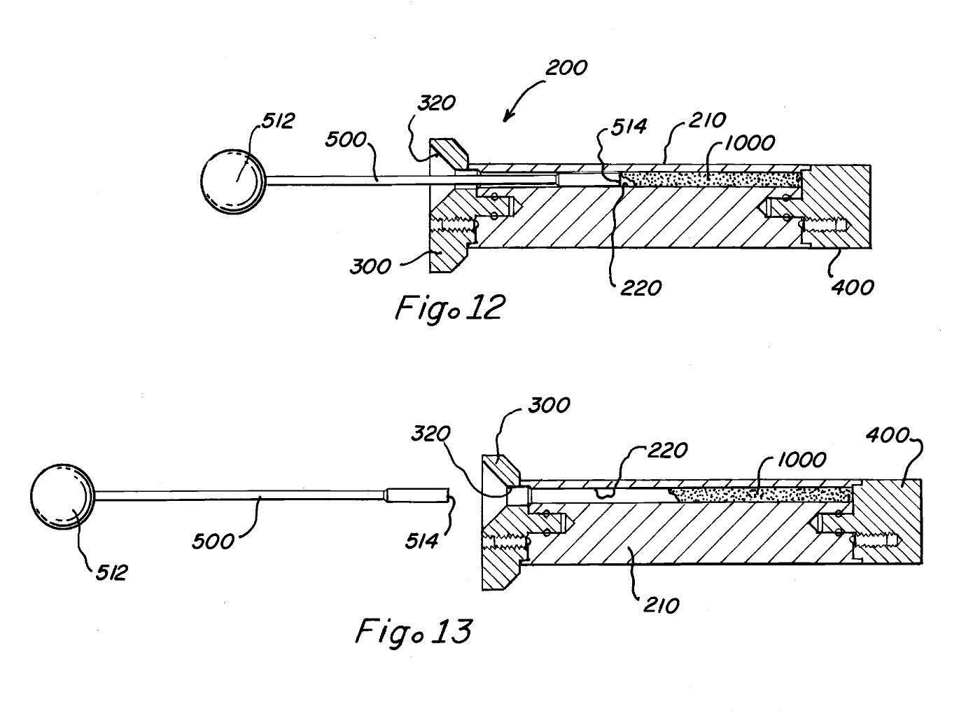

FIG. 12 shows the preloader rod 500 after it has been passed through the loading opening 320 and into the opening 220. The preloader rod 500 or plunger is used to compress the bone graft material 1000 down through the loading opening 320 and into the elongated chamber 220 of the body 210 of the preloader assembly 200. Bone graft material 1000 is added and compressed until the selected loading chamber or elongated passage 220 of the body 210 is filled to a selected level from the distal end 214 to a position near the proximal end 212 of the body 210 of the elongated passage 220. The first end cap 300 and the loading opening 320 and associated funnel 322 may then be rotated to align to the next elongate passage 222 in the body 210 of the preloading assembly 200. The steps as described above in this paragraph may be repeated until each of the plurality of elongated passages or chambers are subsequently filled in this same manner. It should be noted that in some instances only one elongated passageway may be filled and compacted. In other instances, fewer than all of the elongated passages may be filled in the manner described above. Of course, if another chamber or elongated passageway is to be filled and compacted, the preloader rod 500 has to be removed and reused on each chamber or elongated passageway.

An embodiment of the preloader rod 500 may include a vent so that compacting the bone graft material 912 may also include removing air, vapor or other gases from the bone graft material. The preloader rod 500 may include a feature, such as a groove, that serves as a vent to allow air, vapor or other gases to be expelled through the groove. In other embodiments, the outside diameter of the distal end 514 of the preloader rod 500 is dimensioned to allow some if not substantially all of the air, vapor or other gases to be removed during compaction of the bone graft materials 1000 in the chamber or elongated passage 220.

FIG. 13 is a cross sectional view of the preloader assembly 200 having a chamber or elongated passage containing compacted bone graft material 1000 with the second end cap 400 in a closed position. The bone graft material 1000 is compacted against the surface of the end cap 400 and forms a compacted volume from the second end 214 of the body 210 of the preloader to a selected point between the first end 212 and the second end 214 of the body 210 in the elongated passage or chamber 220. The preloader rod 500 is shown removed from the loading opening 320 and the elongated passage or chamber 220.

Transferring the compacted volume of bone graft material 1000 includes rotating or otherwise moving the second end cap 400 positioned at a second end of the preloader body 210 from a closed position to an open position. In the open position, the flat portion of the second end cap that covered the chamber or elongated passage 220 is moved out of the way and the extraction opening 420 is substantially aligned with elongated passage or chamber 220 of the body 210 of the preloader assembly 200. The dispenser assembly 600 is then engaged with the second end cap 400.

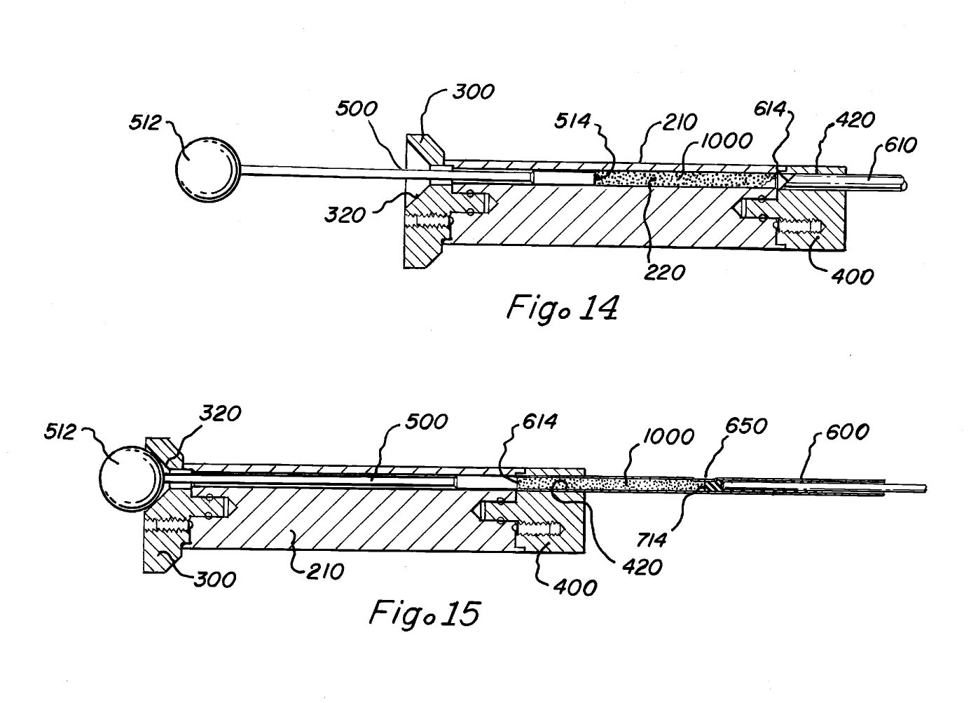

FIG. 14 is a cross sectional side view showing the second end cap 400 in the open position with the distal portion of the dispenser system 600 engaged with the extraction opening 420. The dispenser rod 700 is placed in the load position where the projection 740 is stopped or positioned against the proximal stop 642 in the guide 640 (shown in FIG. 4). Only a portion of the dispenser assembly 600 is shown in FIG. 14. The distal end 614 of the cannula 610 is placed into the extraction opening 420 of the second end cap 400. The extraction opening 420 is a passage and acts as a link to temporarily mate the distal end 614 of the cannula 610 of the dispenser system 600 to the elongated passage 220 holding compacted bone graft material 1000. The inside diameter of the extraction opening 420 accommodates the outside diameter of the cannula 610. The inside diameter of the cannula 610 or diameter of the lumen 622 is about the same as the diameter of the elongated passage 220 of the body 210. The preloader rod 500 is then used to engage and push the compacted bone graft material 1000 from the elongated passage 220, through the passage 420 and into the lumen 622 of the cannula 610 of the dispenser assembly 600. Placing the distal end 614 of the cannula 610 into the extraction opening 420 of the second end cap 400 can also be thought of as moving the cannula 610 to a transfer position with respect to the second end cap 400.

FIG. 15 is a cross sectional side view showing the preloader rod 500 pushing the volume of bone graft material 1000 through the second end cap 400 and into distal portion of the dispenser system 600 engaged with the extraction opening 420. As shown in FIG. 15, the dispenser rod 700 is within the cannula 610, thereby forming the dispenser system 600 (shown in FIG. 1). The dispenser rod 700 is in the loading position.

FIG. 16 is a top view of the vent 650 and the distal end 714 of the dispenser rod 700 when the dispenser rod 700 is in the loading position. When the dispenser rod 700 is in the loading position (where the projection 740 is at the proximal end stop 642, as shown in FIG. 4) the distal end 714 of the dispenser rod 700 is positioned near the distal end of the vent 650 in the cannula 610. The distal end 714 of the dispenser rod 700 covers enough of the vent 650 to prevent the bone graft material 1000 from exiting the vent opening 650. The distal end 714 of the dispenser rod 700 is positioned so that it leaves enough of an opening to allow air, vapor or other gases, and the like to pass from the compacted bone graft material 1000 through the vent 650. The placement by design of the vent at a distance proximal from the distal end 614 of the cannula 610 is determined by the volume of bone graft material 1000 being dispensed from one elongated passage 220 or chamber in the body 210 into the distal end 614 of the dispenser cannula 614, in a manner that will be described later, below.

Further venting may occur when using some of the embodiments of the dispenser rod 700. As described in FIG. 5, one embodiment of the dispenser rod 700 has a tip 720 with a dispenser vent 726 formed in an surface of the tip 720. As illustrated, the dispenser vent 726 is configured as a groove. The dispenser vent 726 serves as a vent to allow air, vapor or other gases to be expelled through the groove as illustrated. Therefore, when the preloader rod is transferring the volume of bone graft material 1000 into the dispenser system 600, the last part of the transfer will cause one end of the volume of bone graft material 1000 to contact and compress against the distal end 714 of the dispenser rod 700. The tip 720 of the dispenser rod 700 functions like a piston. The dispenser vent 726 in the tip 720 located at the distal end 714 of the dispenser rod will also vent air, vapor or other gases from the compacted volume of bone graft material 1000. The vent 650 and dispenser vent 726 will also vent any air, vapor or other gases that may have been otherwise introduced during the transfer process. In some embodiments, preloader rod may be used to compact the material to a selected pressure or to a selected level or volume. As shown in FIG. 15, this selected volume may correspond to the volume between the preloader rod 500 distal ends 514 and the dispenser rod 700 distal end 714. In some embodiments, this volume may also correspond to a selected dosage of bone graft material 1000.

As shown in FIG. 15, the first end cap 300 remains in place during the transfer operation. In some other embodiments, the first end cap 300 is removed to allow the preloader rod 500 and more precisely the distal end 514 of the preloader rod 500 to move the volume of compacted material 1000 further away from the second end 214 of the body 210 containing the elongated passage 220.

FIG. 17 shows a cross sectional side view of the distal end 614 of the dispenser system 600 filled with a volume of compacted bone graft material 1000. Once the bone graft material 1000 has been transferred the distal end 614 of the dispenser system 600 is disengaged with the end cap 400 of the preloader assembly 200.

It should be noted that in some embodiments, the volume of the elongated passage and more specifically the volume of the bone graft material 1000 within the passage 220 is selected to be substantially equal to the volume of the dispenser system 600 between the distal end 614 of the cannula 610 and the distal end 714 of the dispenser rod 700. This fills the cannula volume and assures that the compacted bone graft material 1000 engages the distal end of the dispenser rod 700. This also assures a known volume of the bone graft material 1000 that can represent a dose or known amount, thus eliminating or reducing the amount of guesswork in determining if the intervertebral space has been filled to a desired level while dispensing the bone graft material 1000.

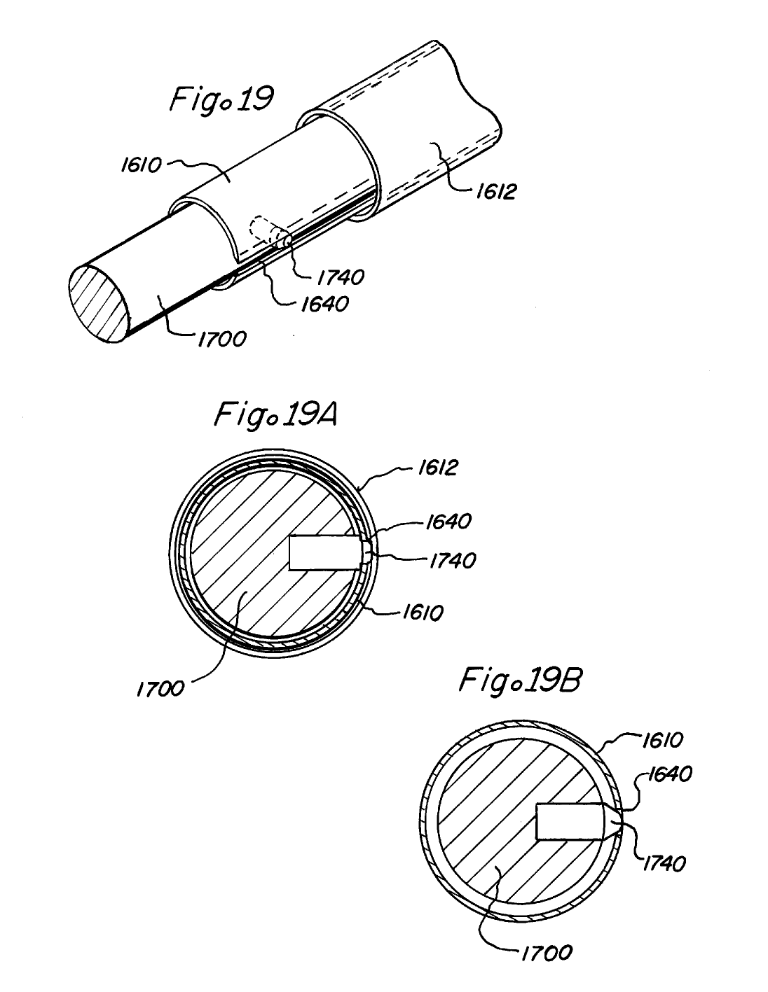

FIG. 18 shows a cross sectional side view of the dispenser system 600 having the distal end 614 of the dispenser system 600 positioned within the intervertebral cavity and having partially dispensed a portion of compacted bone graft material 1000, according to an exemplary embodiment. Positioning the distal end 614 of the cannula 610 to a selected position 918, includes placing the dispenser assembly within a working cannula (not shown) to place the distal end 614 of the cannula 610 into the intervertebral space. One method for placing the working cannula is for a trans-sacral axial approach (described and disclosed in commonly assigned U.S. Pat. Nos. 6,558,386; 6,558,390; 6,575,979; 6,921,403; 7,014,633, and 7,087,058, the disclosures of each of which are herein incorporated by reference). It should be noted that other approaches may be employed and that the method and system 100 described herein will be similarly effective for dispensing bone graft material. Once in the desired cavity, the distal end 614 can be rotated to a desired position. The rotational position can be determined by placing at least one rotational index on the body of the cannula 610. Generally, this will be placed at or near the proximal end 612 of the cannula 610 to facilitate ease of use by a surgeon. Once placed as desired, the surgeon can dispense the bone graft material. The surgeon can dispense all or part of the volume 1000. This process can be repeated until a desired amount of bone graft material 1000 is dispensed into the intervertebral space.

Dispensing the compacted bone graft material 1000 includes moving the dispenser rod 700 in the guide 640 of the cannula 610. This includes moving the engaged projection 740 of the dispenser rod 740 from the proximal end stop 642 to the distal end stop 644. Dispensing further includes passing any intermediate stops, such as stops 648, 646 between the proximal end stop 642 and the distal end stop 644 (shown in FIG. 5) thereby advancing the dispenser rod 700 distally through the lumen 622 of the cannula until the projection 740 contacts the distal end stop 644. In some applications, the surgeon may decide to cease advancing the dispenser rod 700 before the projection 740 contacts the distal end stop 644, such as when the surgeon has determined that the intervertebral space has been substantially filled. The intermediate stops 646, 648 can correspond to dosage levels or volume levels to aid a surgeon in making this determination. The intermediate stops also serve to slow down the dispensing of the bone graft material.

### ALTERNATIVES AND VARIATIONS

### Delivery of Bone Graft Material to Motion Segment Other than L5/S1

In order to provide concreteness to the disclosure provided above, a specific motion segment was discussed. In this instance it was the L5/S1 motion segment. While the dimensions of components may be slightly different when accessing or treating a different 15 motion segment, nothing in the above disclosure should be interpreted as limiting the disclosure to therapeutic treatment of the L5/S1 motion segment. Other motion segments including by way of example and not limitation are the L5/L4 motion segment and the L3/L4 motion segment which for example, following nucleectomy and fixation in preparation for fusion therapy, may benefit from delivery of bone graft material 1000 via the dispenser system 100 assembly that uses one or more teachings from the present disclosure. For example, for a procedure simultaneously providing spinal fusion therapy to two vertebral levels, e.g., L4/L5 and L5/S1, the cannula 610 of the dispenser system 600 would be provided with bushings sized to fit within a larger diameter working cannula (12 mm) used in that procedure. Moreover, there might be an increase in the diameter of the lumen 622 of the dispenser cannula 610 to allow for greater bone graft material 1000 delivery per loading chamber or elongated passage 220, 222, 224 in the body 210 of the preloader assembly 200t since L4-L5 discs are quite taller than L5-S1 discs, and a procedure on that motion segment will necessitate use of a greater volume of bone graft material.

Moreover, as noted previously, the deployment of bone graft material 1000 via a controlled rate of delivery could also be accomplished by the bone graft inserter assembly 780, having a threaded portion 782. An example of this embodiment is set forth in FIG. 7. As shown in FIG. 7, the dispenser rod 780 engaged the dispenser cannula 680 (need to show in FIG. 7B) by means of threads and the distal advancement of the dispenser rod 780 through the dispenser cannula 680 to deploy the bone graft material 1000 by means of rotation.

### Kits

For the convenience of the surgeons, collections of components for a procedure may be combined together in a kit. More specifically, Kits may be prepared to include the components of the dispenser system 100. For example, a kit could include one or more of the devices discussed and closed above. More specifically, a kit would include at least one of the preloader assembly 200 and the dispenser assembly 600. It is possible that a single kit would have all the components referenced above, or there may be a distinction between provision of kits with re-usable versus accompanying kits with disposable components, which for convenience, would contain, for example, multiple inserter assemblies. In yet another aspect of the disclosure, disposable Kits may be provided which comprise, for example, at least one dispenser rod 700 with a plurality of dispenser cannulae 61. Other kits may accompany or supplement a re-usable Kit and include a preloader body 210; a first end cap 300 which includes the loading opening 320 with funnel 322, and preloader rod 500. Kits may optionally include bone graft extender materials, although selection and provision of specific bone graft materials is generally made by the surgeon according to individual preference.

### General Comments

In the context of the present disclosure, as used herein the term “assembly” refers to instruments and instruments systems which are configured to comprise multiple components, which may or may not be contiguous. It is further understood that individual components may themselves be configured as sub-assemblies, e.g., comprising a plurality of component materials, and that the formation of the components may involve intermediate processes or appliances. It will also be understood that the individual components as well as the assemblies may be fabricated for either single, one time use (disposable), or for re-use following subsequent cleaning and sterilization, and further, may be provided or packaged accordingly in disposable or reusable kits.

It will also be understood that upon formation of assemblies from multiples components and deployment, individual components may or may not remain as discernibly distinct.

In the context of the present disclosure, and relative to the patient, anterior refers to in front of the spinal column (ventral); posterior refers to behind the column (dorsal); cephalad means towards the patient's head (also sometimes “superior,” or distal); caudal refers to the direction or location that is closer to the feet (also sometimes “inferior,” or proximal). In the context of the present disclosure and relative to the user (surgeon or clinician), in general distal refers to that portion of an instrument (e.g., a tip) which in the position of its intended use is more distant from the user, while proximal (e.g., a handle) is closer to the user. An exception is an instrument or assembly which is manipulated at both end times at different times, e.g., the preloader body 210 which receives bone graft materials 1000 in a first end 212 of the body 210 and from which the bone graft materials 1000 are dispensed at the second end 214. As the present disclosure contemplates accessing the various vertebral bodies and intervertebral disc spaces through a preferred approach that comes in from the sacrum and moves towards the head, proximal and distal are defined in context of this channel of approach. Consequently, proximal is closer to the beginning of the channel and thus towards the feet or the surgeon, distal is further from the beginning of the channel and thus towards the head, or more distant from the surgeon. When referencing delivery tools, distal would be the end intended for insertion into the access channel (whether a trans-sacral access channel or an access channel from another route) and proximal refers to the other end, generally the end closer to the handle for the delivery tool.

It will also be understood that the components, in whole or in part, may be designed and fabricated from materials as biomechanically and clinically indicated. Materials selection and device configuration are based on considerations of minimal wear characteristics and minimal cellular reactions and scar tissue maturation. In one aspect of the present disclosure, certain components of the device assemblies and systems of the present disclosure are configured to comprise biocompatible materials and are able to withstand, without wear, 25 multiple cycles or procedures without failing. Towards this end, in yet another aspect of the present disclosure, instruments and fixation devices may undergo appropriate surface pretreatments to preclude or retard wear.

As used herein, the term “biocompatible” refers to an absence of chronic inflammation response or cytotoxicity when or if physiological tissues are in contact with, or exposed to 30 (e.g., wear debris) the materials and devices of the present disclosure.

In addition to biocompatibility, in another aspect of the present disclosure it is preferred that the materials comprising the instrument systems are sterilizable; visible and/or imageable, e.g., fluoroscopically; or via CT (computed tomography), or if via MRI (magnetic resonance imaging), with this last-named imaging technique mandating that materials be substantially free of Fe (iron) or other magnetic materials. In accordance with one aspect of the embodiments described herein, there are provided certain materials which can enhance visualization of implant assembly components and instrumentation for their deployment via radio-imaging (e.g., fluoroscopy). It will be understood that such enhancing materials (e.g., Ta; barium sulfate powders, etc.) may be incorporated into the formation of certain metal or polymeric materials comprised in the device assemblies and/or tools sets used to deploy the devices of the present disclosure. That is, in consideration of contrast, detail, and spatial sensitivity, it is preferred that contrast media (e.g., iodine) or other materials (e.g., Ta; Ti) may be employed in configuring instrumentation when and where needed and appropriate, to supplement or modify radiolucency or radio-opaqueness.

It will also be understood that to enhance performance, the instruments and assemblies comprised in the present disclosure may be surface-treated or coated with suitable biocompatible materials to facilitate ease of deployment, e.g., render them more hydrophilic.

Percutaneous as used in this disclosure simply means through the skin from a paracoccygeal access point on the patient and to the posterior or anterior target point, as in transcutaneous or transdermal, without implying any particular procedure from other medical arts. However, percutaneous access is distinct from a surgical access, and the percutaneous opening in the skin is preferably minimized so that it is less than four centimeters across, preferably less than two centimeters. The percutaneous access pathway is generally axially aligned with the bore extending from the respective anterior or posterior target point through at least one sacral vertebral body and one or more lumbar vertebral body in the cephalad direction as visualized by radiographic or fluoroscopic equipment.

It will be understood that the surgical access can be conducted by methods other than the preferred approach described, including without limitation open surgical procedures from any access orientation, and that each of the therapies to the spine can be conducted on more than one motion segments (e.g., cervical as well as lumbar) traversed by at least one working channel, with deployment of appropriate implants and with post-procedural surgical closure. Only for convenience, the exemplary access by the method, instrumentation, and, e.g., bone graft materials deployment to a single motion segment are described in detail herein, in accordance with the bone graft materials insertion instrument systems and assemblies of the present disclosure.

It will be further understood that the length and dimensions of instruments and 5 components described herein will depend in part on the nature of the treatment procedure (for example, treatment level, e.g., at L3/L4 lumbar motion segment versus L5/S1, or if a thoracic or cervical levels if by a surgical method other than via trans-sacral access) and the physical characteristics of the patient, as well as the construction materials and intended functionality, as will be apparent to those of skill in the art.

The effort to provide one or more tangible examples in order to promote the understanding of the present disclosure should not be misinterpreted as a limitation on the scope of the teachings as set forth in this disclosure. For example, one of skill in the art will appreciate that indicia/landmarks (e.g., the alignment of the second end cap 400 with the elongated passages 220, 220, 224 of the preloader assembly 200 with an engagement screw with the channel selector dispenser cannula as indicating the closed position re access to loading chambers) could be implemented by various other alternative means not shown.

One of skill in the art will recognize that in some cases that the order of the elements in the method claims may be changed without departing from the teachings of this disclosure.

One of skill in the art will recognize that alternative implementations set forth above are not universally mutually exclusive and that in some cases additional implementations can be created that implement two or more of the variations described above. In a like manner, one of skill in the art will recognize that certain aspects of the present disclosure can be implemented without implementing all of the teachings illustrated in any of the various disclosed implementations. Such partial implementations of the teachings of the present disclosure fall within the teachings of the subject matter unless explicitly calling for the presence of additional elements from other teachings.

In order to promote clarity in the description, common terminology for components is used. The use of a specific term for a component suitable for carrying out some purpose within the disclosed teachings should be construed as including all technical equivalents which operate to achieve the same purpose, whether or not the internal operation of the named component and the alternative component use the same principles. The use of such specificity to provide clarity should not be misconstrued as limiting the scope of the disclosure to the named component unless the limitation is made explicit in the description or the claims that follow.

Similarly, as used herein, some terminology for components are interchangeable (e.g., chamber and elongated passage and channel; guidewire and guide pin and pin; push rod and preloader rod and plunger rod, etc.).

In order to make it easier for a reader to find certain sections of this document that are of particular interest to the reader, a series of headings have been used. These headings are solely for the purpose of helping readers navigate the document and do not serve to limit the relevance of any particular section to exclusively the topic listed in the heading.

Those skilled in the art will recognize that the methods and apparatus of the present disclosure have many applications and that the present teachings are not limited to the specific examples given to promote understanding of the present disclosure. Moreover, the scope of the present disclosure covers the range of variations, modifications, and substitutes for the system components described herein, as would be known to those of skill in the art.

What I claim is:

1. An apparatus for dispensing bone graft material, comprising:
a cannula including a proximal end and a distal end, a cannula inner surface of the cannula defining a lumen between the proximal end and the distal end, the lumen extending substantially along a longitudinal axis of the cannula, the cannula also includes a guide extending along at least a portion of the cannula inner surface;
at least one portion of the guide including a proximal end stop and a distal end stop, the distal end stop positioned toward the distal end of the cannula from the proximal stop, and
the cannula defining a vent positioned between the distal end stop of the guide and the distal end of the cannula, the vent in communication with the lumen of the cannula and adapted to release gas from the lumen; and
a dispenser rod including an outside diameter that is smaller than the inside diameter of the lumen, the dispenser rod further comprising a projection dimensioned to slidably engage the guide in the cannula, the dispenser rod including a distal end and a proximal end.

2. The apparatus of claim 1 further comprising at least one portion of the guide extending along at least substantially parallel to the longitudinal axis of the cannula.

3. The apparatus of claim 1 further comprising the guide of the cannula extending through the inner surface of the cannula to form a slot.

4. The apparatus of claim 3 wherein the projection is slidably received in the slot.

5. The apparatus of claim 3 wherein the projection extends into the slot, the end of the projection located between the lumen and the outer surface of the cannula.

6. The apparatus of claim 1 further comprising the guide configured as a channel on the inner surface of the cannula and at least a portion of the projection slidably received within the channel.

7. The apparatus of claim 1 further comprising the guide defining at least one intermediate stop between the proximal end stop and the distal end stop.

8. The apparatus for dispensing bone graft material of claim 1 wherein the distal end of the cannula is beveled.

9. The apparatus for dispensing bone graft material of claim 1 wherein the dispenser rod includes a piston secured to the distal end of the dispenser rod, the piston substantially cylindrically shaped and including a proximal end, a distal end, and an outside diameter that is smaller than the inside diameter of the lumen of the cannula, the lumen defining a round cross sectional shape in a transverse plane.

10. The apparatus for dispensing bone graft material of claim 9 further comprising the piston defining a dispensing vent.

11. The apparatus for dispensing bone graft material of claim 1 wherein the distal end of the dispenser rod is positioned within the lumen near one edge of the vent, when the projection of the dispenser rod is positioned against the proximal end stop of the guide of the cannula.

12. The apparatus for dispensing bone graft material of claim 1 wherein the distal end of the dispenser rod is positioned within the lumen and covering the vent, when the projection of the dispenser rod is positioned against the proximal end stop of the guide of the cannula.

13. The apparatus for dispensing bone graft material of claim 10 wherein the volume of the lumen between the distal end of the dispenser rod when in a load position, and the distal end of the cannula defines a dispensing volume of bone graft material.

14. The apparatus for dispensing bone graft material of claim 1 wherein the cannula includes graduation marks in the cannula that correspond to volume levels associated with the portion of the cannula between the distal end of the cannula and the distal end of the dispenser rod.

15. The apparatus for dispensing bone graft material of claim 1 wherein the guide includes at least one intermediate stop.

16. The apparatus for dispensing bone graft material of claim 1 wherein the guide includes a plurality of intermediate stops between the proximal end stop and the distal end stop.

17. The apparatus for dispensing bone graft material of claim 16 wherein the intermediate stops correlate to volumes in the lumen between the distal end of the cannula and the distal end of the dispenser rod.

18. A kit for loading a bone graft material into an insertion cannula, comprising:
a preloading assembly, comprising a body defining a first end, a second end, and a plurality of elongated passages extending between the first end and the second end, a first end cap secured to a first end of the body over at least one of the plurality of elongated passages, the first end cap defining a loading opening, the loading opening positionable over at least one of the plurality of openings and adapted to receive a bone graft material and to transfer the bone graft material into at least one of the plurality of elongated passages, and a second end cap movably secured to a second end of the body, the second end cap defining an extraction opening, the second end cap movable between at least one closed position and at least one open position with respect to at least one of the plurality of elongated passages, the extraction opening in the at least one open position in communication with at least one of the plurality of elongated passages; and
a preloader rod dimensioned to fit in a portion of the at least one of the plurality of elongated passages.

19. The kit of claim 18, the preloading assembly including a at least two elongated passage defining a circular transverse cross-section along at least a portion of a length of the at least two elongated passages.

20. The kit of claim 18, the preloading assembly further comprising at least a portion of the body formed from a translucent material, the translucent material extending along at least a portion of the length of at least one of the plurality of elongated passages, the translucent material adapted to transmit light between an inner surface of the body defining at least one of the elongated passages and an outer surface of the body to visualize a volume of a bone graft material positioned within the passage.

21. The kit of claim 20, the preloading assembly wherein the body includes one or more graduations on an outer surface of the body and extending along at least a portion of the length of at least one of the plurality of elongated passages to indicate the volume of bone graft material in the elongated passages.

22. The kit of claim 18, further comprising at least a portion of the preloader rod defining an outside diameter that slidably engages an inside diameter of at least one of the plurality of elongated passages.

23. The kit of claim 18, further comprising the preloader rod further comprising a plunger secured to a distal end of the preloader rod, the plunger defining an outside diameter adapted to slidably engage an inside diameter of the at least one of the plurality of elongated passages of the body.

24. The kit of claim 23, further comprising the plunger of the preloader rod further defining a plunger vent adapted to vent a gas between a first plunger end and a second plunger end.

25. The kit of claim 24 wherein the plunger is cylindrically shaped and defines a longitudinal axis, and wherein the plunger vent is configured as a groove along an outer plunger surface of the plunger between the first plunger end and the second plunger end.

26. The kit of claim 18, wherein the first end cap defines a funnel adjacent to the loading opening, the funnel adapted to guide the bone graft material into the loading opening.

27. The kit of claim 26 further comprising the first end cap rotatably secured to the first end of the body, the first end cap being rotatable with respect to the first end of the body such that the loading opening in the first end cap can be aligned with at least one of the plurality of elongated passages of the body.

28. The kit of claim 20 wherein the extraction opening in the second end cap defines a cylindrical shape along at least a portion of an extraction opening length, the cylindrical shape including substantially the same inner diameter as the inner diameter of the at least one passage of the body, the extraction opening of the second end cap substantially aligning with the at least one passage of the body in the open position.

29. The kit of claim 27, the second end cap rotatably movable about a longitudinal axis of the body between the open position and the closed position.

30. A method for dispensing bone graft material, comprising:
placing the bone graft material into an elongated chamber defined in a preloading assembly;
compacting the material in the chamber;
transferring the compacted material in the chamber to a volume in a distal end of a cannula of a dispenser apparatus;
venting compressed air in the cannula from the compacted material through a vent in the cannula;
positioning the distal end of the cannula to a selected position; and
dispensing at least a portion of the material to be dispensed by moving an dispenser rod through the cannula.

31. The method of claim 30 further comprising engaging a projection of a dispenser rod into a guide defined in the cannula to provide at least a distal end stop, and the dispensing further comprising advancing the dispenser rod distally through the lumen of the cannula until the projection contacts the distal end stop.

32. The method of claim 30 further comprising engaging a projection of a dispenser rod into a guide defined in the cannula and providing at least an intermediate stop and a distal end stop positioned along the guide, and the dispensing further comprising advancing the dispenser rod distally through the lumen of the cannula until the projection contacts the intermediate stop to temporarily stop the longitudinal advancing of the dispenser rod; rotating the dispenser rod about a longitudinal axis of the dispenser rod to disengage the projection from the intermediate stop, and advancing the dispenser rod distally through the lumen of the cannula until the projection contacts the distal end stop.

33. The method of claim 30 wherein compacting the material in a chamber in the preloading assembly includes using a preloader rod to compact the material to substantially remove air vapor from the material.

34. The method of claim 33 wherein the materials include a slurry of bone graft material.

35. The method of claim 30 wherein compacting the material in a chamber in the preloading assembly includes using a preloader rod to compact the material to a selected volume.

36. The method of claim 30 wherein compacting the material in a chamber in the preloading assembly includes using a preloader rod to compact the material to a selected volume, so that a selected dosage of material is associated with the chamber in the preloader.

37. The method of claim 30 wherein placing the material into the chamber of the preloading assembly includes aligning a funnel-shaped opening in a first end cap with the chamber of the preloader assembly.

38. The method of claim 37 wherein compacting the material in a chamber in the preloading assembly includes passing a preloader rod to compact the material through the funnel shaped opening in the first end cap.

39. The method of claim 38 therein the preloader assembly includes a second end cap positioned at a second end of the preloader assembly in a closed position, wherein the second end cap closes the chamber in the preloading assembly.

40. The method of claim 39 wherein transferring the compacted material in the chamber to a volume in a distal end of a cannula of a dispenser apparatus further comprises:
moving the second end cap to an open position with respect to the chamber in the preloader assembly;
moving the cannula to a transfer position with respect to the second end cap; and
pushing the compacted material from the chamber into the cannula with the preloader rod.

41. The method of claim 40 further wherein pushing the compacted material into the cannula includes venting air from the compacted material.

42. The method of claim 40 wherein the chamber of the preloader assembly is an elongated cylindrical and wherein the opening in the second end cap is a cylinder including substantially the same inner diameter as the inner diameter of the chamber.

43. The method of claim 42 wherein the cannula includes an inside diameter substantially the same as the inside diameter of the opening in the second end cap.

44. An apparatus for loading a bone graft material into an insertion cannula, comprising:
a preloading assembly, comprising a body defining first end, a second end, and a plurality of elongated passages extending between the first end and the second end, a first end cap secured to a first end of the body over at least one of the plurality of elongated passages, the first end cap defining a loading opening, the loading opening positionable over at least one of the plurality of openings and adapted receive a bone graft material and to transfer the bone graft material into at least one of the plurality of elongated passages, and a second end cap movably secured to a second end of the body, the second end cap defining an extraction opening, the second end cap movable between at least a closed position and at least one open position with respect to at least one of the plurality of elongated passages, the extraction opening in the at least one open position in communication with at least one of the plurality of elongated passages; and
a preloader rod dimensioned to fit in a portion of the at least one of the plurality of elongated passages;

45. The apparatus of claim 44, the preloading assembly including at least two elongated passages defining a circular transverse cross-section along at least a portion a length of the at least two elongated passages.

46. The apparatus of claim 44, the preloading assembly further comprising at least a portion of the body formed from a translucent material, the translucent material extending along at least a portion of the length of at least one of the plurality of elongated passages and adapted to transmit light between an inner surface of the body defining at least one of the elongated passages and an outer surface of the body.

47. The apparatus of claim 44, further comprising the preloader rod defining an outside diameter that fits within the inside diameter of the cylindrical opening.

48. The apparatus of claim 44, further comprising the preloader rod further comprising a plunger end including outside diameter adapted to slidably engage the inside diameter of the at least one of the plurality of elongated passages of the body.

49. The apparatus of claim 48, further comprising the plunger end of the preloader rod further defining a plunger vent adapted to allow gas to pass between a first plunger end and a second plunger end.

50. The apparatus of claim 49 wherein the plunger is cylindrically shaped and defines a longitudinal axis, and wherein the plunger vent is configured as a groove along an outer plunger surface of the plunger between the first plunger end and the second plunger end.

51. The apparatus of claim 44, wherein the first end cap defines a funnel adjacent to the loading opening, the funnel adapted to guide the bone graft material into the loading opening.

52. The apparatus of claim 51 further comprising the first end cap rotatably secured to the first end of the body, the first end cap being rotatable with respect to the first end of the body such that the loading opening in the first end cap is aligned with at least one of the plurality of elongated passages of the body.

53. The apparatus of claim 46 wherein the extraction opening in the second end cap defines a cylindrical shape along at least a portion of an extraction opening length, the cylindrical shape including substantially the same inner diameter as the inner diameter of the at least one passage of the body, the extraction opening of the second end cap substantially aligning with the at least one passage of the body in the open position.

54. The apparatus of claim 53 the second end cap covers all of the passages of the body when in the closed position.

55. The apparatus of claim 54, wherein the second end cap is rotatably movable about a longitudinal axis of the body between the open position and the closed position.
